(19) United States
(12) Patent Application Publication
(10) Pub. No.: US 2014/0271453 A1
(43) Pub. Date: Sep. 18, 2014

(54) METHODS FOR THE EARLY DETECTION OF LUNG CANCER

(71) Applicant: Abbott Laboratories, Abbott Park, IL (US)

(72) Inventors: Barry L. Dowell, Mundelein, IL (US); Philip M. Hemken, Pleasant Prairie, WI (US)

(21) Appl. No.: 13/804,639

(22) Filed: Mar. 14, 2013

(51) Int. Cl. G01N 33/574

(52) U.S. Cl. CPC G01N 33/57423
USPC 424/1.11; 435/7.6

(57) ABSTRACT

Provided are methods of diagnosing lung cancer, particularly at an early stage, such as stage I, by detecting the presence and/or amount of at least four biomarkers of lung cancer in a sample from a subject as well as determining the number of smoking pack years for the subject. The methods and biomarkers may be used to develop an accurate prognosis for a patient having lung cancer or suspected of having lung cancer or accurately diagnose a subject having or suspected of having lung cancer. The methods and biomarkers may be used to identify and/or classify a patient as a candidate for lung cancer treatment therapy.

### FIELD OF THE INVENTION

The present invention relates to methods and immunoassay platforms for determining a prognosis, diagnosis or risk identification of lung cancer in a patient by detecting biomarkers in the patient as well as determining the number of pack years of smoking of the patient. The biomarkers and number of pack years of smoking of the patient can be used to identify or diagnosis a patient with lung cancer, particularly, early stage lung cancer, to identify whether a patient is suffering from lung cancer or benign lung disease, to identify a patient as a candidate for a lung cancer treatment regimen, to classify a patient's risk of developing lung cancer, as well as to determining a diagnosis, prognosis or treatment regimen.

### BACKGROUND

Globally, lung cancer is the leading cause of cancer-related death in males and the second leading cause in females accounting for 1.4 million lung cancer deaths per year. Despite treatment advances, survival has not improved substantially over the past 30 years, mainly because the majority of the patients have distant metastases at the time of diagnosis. (See, Horeweg et al., Am J Respir Crit. Care Med., (Jan. 24, 2013)).

In 2012, it was estimated that 160,300 deaths (87,700 men and 72,600 women) from lung cancer would occur in the United States. The 5-year survival rates for lung cancer are about 15.6%, in part because most patients have advanced stage lung cancer at their initial diagnosis. (http://seer.cancer.gov/statfacts/html/lungb.html). Thus, early detection of lung cancer is an important opportunity for decreasing mortality. Considerable interest has been shown in developing tools to detect early stage lung cancer. Recent data suggests using low dose computed tomography (CT) scans to screen patients who are at high risk for lung cancer. Depending on the results, such individuals would be subjected to additional imaging with positron emission tomography (PET) or would undergo a biopsy in order to establish a diagnosis (See, NCCN Guidelines Version 1.2013 Lung Cancer Screening).

Biomarker analysis is potentially one element of such a diagnostic strategy, but the discovery and validation of tumor specific markers has been difficult. Currently, the focus is on using a combination of biomarkers plus nodule size based on imaging, to stratify patients as to the risk of lung cancer (i.e., low-risk or high-risk). (Patz et al., Am J Respir Crit. Care Med. (January 2013). Accordingly, there remains a great need for more accurate and precise markers for the detection of lung cancer, particularly early stage lung cancer.

### SUMMARY OF THE INVENTION

The present invention is directed to a method of identifying and treating a subject having or at risk of having lung cancer, the method comprising the steps of: obtaining a biological sample from a subject; determining the levels of midkine (MK), tissue factor pathway inhibitor (TFPI), neuron-specific enolase (NSE) and carbohydrate antigen 19-9 (CA19-9) in the biological sample from the subject; determining the number of pack years of smoking of the subject; comparing the levels of MK, TFPI, NSE and CA19-9 in the biological sample to reference levels of MK, TFPI, NSE and CA19-9; comparing the number of pack years of smoking by the subject to a reference level of pack years; identifying the subject as having lung cancer or at risk of having lung cancer if the levels of MK, TFPI, NSE and CA19-9 in the biological sample are greater than the reference levels of MK, TFPI, NSE and CA19-9 and the number of pack years of smoking by the subject is greater than the reference level of pack years; and administering a lung cancer treatment regimen to the subject identified as having lung cancer or a lung monitoring regimen to the subject identified as at risk of having lung cancer.

The lung cancer identified pursuant to the above method may be early lung cancer (or early stage lung cancer). For example, the early lung cancer may be Stage I or Stage II non-small cell lung cancer or limited stage small cell lung cancer. The reference levels of MK, TFPI, NSE and CA19-9 may be the MK, TFPI, NSE and CA19-9 cutoff values determined by a receiver operating curve (ROC) analysis from biological samples of a patient group. Alternatively, the reference levels of MK, TFPI, NSE and CA19-9 are the MK, TFPI, NSE and CA19-9 cutoff values may be determined by a quartile analysis of biological samples of a patient group. The MK, TFPI, NSE and CA19-9 reference level may be higher than or equal to 0.05 ng/mL, 0.06 ng/mL, 0.07 ng/mL, 0.08 ng/mL, 0.09 ng/mL, 0.10 ng/mL, 0.20 ng/mL, 0.30 ng/mL or 0.40 ng/mL in serum for MK in combination with levels higher than or equal 20 pg/mL, 21 pg/mL, 22 pg/mL, 23 pg/mL, 24 pg/mL, 25 pg/mL, 26 pg/mL, 27 pg/mL, 28 pg/mL, 29 pg/mL, 30 pg/mL, 31 pg/mL, 32 pg/mL, 33 pg/mL, 34 pg/mL, 35 pg/mL, 36 pg/mL, 37 pg/mL, 38 pg/mL, 39 pg/mL or 40 pg/mL in serum for TFPI, levels higher than or equal to 1 ng/mL, 2 ng/mL, 3 ng/mL, 4 ng/mL or 5 ng/mL in serum for NSE and levels higher than or equal to 10 U/mL, 11 U/mL, 12 U/mL, 13 U/mL, 14 U/mL, 15 U/mL, 16 U/mL, 17 U/mL, 18 U/mL, 19 U/mL, 30 U/mL, 31 U/mL, 32 U/mL, 33 U/mL, 34 U/mL, 35 U/mL, 36 U/mL or 37 U/mL in serum for CA19-9.

The method may comprise determining the level of at least one additional biomarker of lung cancer in the biological sample and comparing the level of the at least one additional biomarker of lung cancer to a reference level for the at least one biomarker of lung cancer. Additional biomarkers that may be determined may be selected from the group consisting of: nectin-4, cytokeratin 19-fragment 21-1 (CYFRA 21-1), carcinoembryonic antigen (CEA), progastrin releasing peptide (proGRP), carbohydrate antigen 125 (CA125), tissue polypeptide specific antigen (TPS), cancer antigen 15-3 (CA15-3), squamous cell carcinoma antigen (CCA), Helicobacter pylori, parainfluenza and combinations thereof.

The subject in the method may be a human. The biological sample obtained from the subject may be selected from a tissue sample, bodily fluid, whole blood, plasma, serum, urine, bronchoalveolar lavage fluid, and a cell culture suspension or fraction thereof. For example, the biological sample of the subject may be a blood plasma or blood serum.

The levels of MK, TFPI, NSE and CA19-9 may be determined by an immunological method using one or more molecules that specifically binding to MK, TFPI, NSE or CA19-9. For example, a molecule that specifically binds to MK, TFPI, NSE or CA19-9 is at least one antibody that is capable of specifically binding MK, TFPI, NSE or CA19-9.

In the method, if the levels of MK, TFPI, NSE or CA19-9 are above the cutoff values, this indicates the subject is suffering from lung cancer. In the method, if the number of pack years is above the reference level, this indicates that the subject is suffering from lung cancer.

The lung cancer treatment regimen in the method may comprise administering at least one of surgery, radiotherapeutic therapy, radiotherapeutic treatments, chemotherapy, targeted therapy or combinations thereof to the subject. The lung monitoring regimen may comprise at least one of conducting a chest computed tomography (CT) scan, positron emission tomography (PET) scan, measuring lung function and determining MK, TFPI, NSE and CA19-9 levels at periodic intervals.

In the method, determining the level of MK, TFPI, NSE and CA19-9 may involve the step of contacting the biological sample with at least one antibody selected from the group consisting of: an antibody that specifically binds to MK, an antibody that specifically binds to TFPI, an antibody that specifically binds to NSE, an antibody that specifically binds to CA19-9 and combinations thereof.

In the method, determining the level of MK, TFPI, NSE and CA19-9 may involve the step of assaying the biological sample for MK, TFPI, NSE and CA19-9 by an immunoassay that employs at least one capture antibody and at least one antibody labeled with a detectable label, which generates a signal, and comprises comparing a signal generated by the detectable label as a direct or indirect indication of the amount of MK, TFPI, NSE and CA19-9 in the biological sample, wherein the capture antibody and the antibody labeled with a detectable label comprise:

a. at least one capture antibody that specifically binds to MK and at least one antibody labeled with a detectable label;

b. at least one capture antibody that specifically binds to TFPI and at least one antibody labeled with a detectable label;

c. at least one capture antibody that specifically binds to NSE and at least one antibody labeled with a detectable label; and

d. at least one capture antibody that specifically binds to CA19-9 and at least one antibody labeled with a detectable label.

The immunological method used in the method may comprise:

(a) measuring the levels of MK by:
(i) contacting the test sample with at least one capture antibody, wherein the capture antibody binds to an epitope on MK or a fragment of MK to form a capture antibody-MK antigen complex;
(ii) contacting the capture antibody-MK antigen complex with at least one detection antibody comprising a detectable label, wherein the detection antibody binds to an epitope on MK that is not bound by the capture antibody and forms a capture antibody-MK antigen-detection antibody complex; and
(iii) determining the MK levels in the test sample based on the signal generated by the detectable label in the capture antibody-MK-9 antigen-detection antibody complex formed in (a)(ii);

(b) measuring the levels of TFPI by:
(i) contacting the test sample with at least one capture antibody, wherein the capture antibody binds to an epitope on TFPI or a fragment of TFPI to form a capture antibody-TFPI antigen complex;
(ii) contacting the capture antibody-TFPI antigen complex with at least one detection antibody comprising a detectable label, wherein the detection antibody binds to an epitope on TFPI that is not bound by the capture antibody and forms a capture antibody-TFPI antigen-detection antibody complex; and
(iii) determining the TFPI levels in the test sample based on the signal generated by the detectable label in the capture antibody-TFPI antigen-detection antibody complex formed in (b)(ii);

(c) measuring the levels of NSE by:
(i) contacting the test sample with at least one capture antibody, wherein the capture antibody binds to an epitope on NSE or a fragment of NSE to form a capture antibody-NSE antigen complex;
(ii) contacting the capture antibody-NSE antigen complex with at least one detection antibody comprising a detectable label, wherein the detection antibody binds to an epitope on NSE that is not bound by the capture antibody and forms a capture antibody-NSE antigen-detection antibody complex; and
(iii) determining the NSE levels in the test sample based on the signal generated by the detectable label in the capture antibody-NSE antigen-detection antibody complex formed in (c)(ii); and

(d) measuring the levels of CA19-9 by:
(i) contacting the test sample with at least one capture antibody, wherein the capture antibody binds to an epitope on CA19-9 or a fragment of CA19-9 to form a capture antibody-CA19-9 antigen complex;
(ii) contacting the capture antibody-CA19-9 antigen complex with at least one detection antibody comprising a detectable label, wherein the detection antibody binds to an epitope on CA19-9 that is not bound by the capture antibody and forms a capture antibody-CA19-9 antigen-detection antibody complex; and
(iii) determining the CA19-9 levels in the test sample based on the signal generated by the detectable label in the capture antibody-CA19-9 antigen-detection antibody complex formed in (d)(ii).

In the method, the antibody may be selected from the group consisting of: a polyclonal antibody, a monoclonal antibody, a human antibody, an immunoglobulin molecule, a disulfide linked Fv, a monoclonal antibody, an affinity matured, a scFv, a chimeric antibody, a single domain antibody, a CDR-grafted antibody, a diabody, a humanized antibody, a multispecific antibody, a Fab, a dual specific antibody, a DVD, a Fab′, a bispecific antibody, a F(ab′)2, and a Fv.

The present invention is also directed to a method of providing a diagnosis of a subject having lung cancer, the method comprising the steps of: obtaining a biological sample from a subject; determining the levels of midkine (MK), tissue factor pathway inhibitor (TFPI), neuron-specific enolase (NSE) and carbohydrate antigen 19-9 (CA19-9) in the biological sample from the subject; determining the number of pack years of smoking of the subject; comparing the levels of MK, TFPI, NSE and CA19-9 in the biological sample to reference levels of MK, TFPI, NSE and CA19-9; comparing the number of pack years of smoking by the subject to a reference level of pack years; and providing a diagnosis of a subject having lung cancer if the levels of MK, TFPI, NSE and CA19-9 in the biological sample are greater than the reference levels of MK, TFPI, NSE and CA19-9 and the number of pack years of smoking by the subject is greater than the reference level of pack years.

The lung cancer identified pursuant to the above method may be early lung cancer (or early stage lung cancer). For example, the early lung cancer may be Stage I or Stage II non-small cell lung cancer or limited stage small cell lung cancer.

In the method, determining the level of MK, TFPI, NSE and CA19-9 may involve the step of contacting the biological sample with at least one antibody selected from the group consisting of: an antibody that specifically binds to MK, an antibody that specifically binds to TFPI, an antibody that specifically binds to NSE, an antibody that specifically binds to CA19-9 and combinations thereof.

In the method, determining the level of MK, TFPI, NSE and CA19-9 may involve the step of assaying the biological sample for MK, TFPI, NSE and CA19-9 by an immunoassay that employs at least one capture antibody and at least one antibody labeled with a detectable label, which generates a signal, and comprises comparing a signal generated by the detectable label as a direct or indirect indication of the amount of MK, TFPI, NSE and CA19-9 in the biological sample, wherein the capture antibody and the antibody labeled with a detectable label comprise:

a. at least one capture antibody that specifically binds to MK and at least one antibody labeled with a detectable label;

b. at least one capture antibody that specifically binds to TFPI and at least one antibody labeled with a detectable label;

c. at least one capture antibody that specifically binds to NSE and at least one antibody labeled with a detectable label; and

d. at least one capture antibody that specifically binds to CA19-9 and at least one antibody labeled with a detectable label.

The method may comprise an immunological method which comprises:
(a) measuring the levels of MK by:
   (i) contacting the test sample with at least one capture antibody, wherein the capture antibody binds to an epitope on MK or a fragment of MK to form a capture antibody-MK antigen complex;
   (ii) contacting the capture antibody-MK antigen complex with at least one detection antibody comprising a detectable label, wherein the detection antibody binds to an epitope on MK that is not bound by the capture antibody and forms a capture antibody-MK antigen-detection antibody complex; and
   (iii) determining the MK levels in the test sample based on the signal generated by the detectable label in the capture antibody-MK-9 antigen-detection antibody complex formed in (a)(ii);
(b) measuring the levels of TFPI by:
   (i) contacting the test sample with at least one capture antibody, wherein the capture antibody binds to an epitope on TFPI or a fragment of TFPI to form a capture antibody-TFPI antigen complex;
   (ii) contacting the capture antibody-TFPI antigen complex with at least one detection antibody comprising a detectable label, wherein the detection antibody binds to an epitope on TFPI that is not bound by the capture antibody and forms a capture antibody-TFPI antigen-detection antibody complex; and
   (iii) determining the TFPI levels in the test sample based on the signal generated by the detectable label in the capture antibody-TFPI antigen-detection antibody complex formed in (b)(ii);
(c) measuring the levels of NSE by:
   (i) contacting the test sample with at least one capture antibody, wherein the capture antibody binds to an epitope on NSE or a fragment of NSE to form a capture antibody-NSE antigen complex;
   (ii) contacting the capture antibody-NSE antigen complex with at least one detection antibody comprising a detectable label, wherein the detection antibody binds to an epitope on NSE that is not bound by the capture antibody and forms a capture antibody-NSE antigen-detection antibody complex; and
   (iii) determining the NSE levels in the test sample based on the signal generated by the detectable label in the capture antibody-NSE antigen-detection antibody complex formed in (c)(ii); and
(d) measuring the levels of CA19-9 by:
   (i) contacting the test sample with at least one capture antibody, wherein the capture antibody binds to an epitope on CA19-9 or a fragment of CA19-9 to form a capture antibody-CA19-9 antigen complex;
   (ii) contacting the capture antibody-CA19-9 antigen complex with at least one detection antibody comprising a detectable label, wherein the detection antibody binds to an epitope on CA19-9 that is not bound by the capture antibody and forms a capture antibody-CA19-9 antigen-detection antibody complex; and
   (iii) determining the CA19-9 levels in the test sample based on the signal generated by the detectable label in the capture antibody-CA19-9 antigen-detection antibody complex formed in (d)(ii).

In the method, the antibody may be selected from the group consisting of: a polyclonal antibody, a monoclonal antibody, a human antibody, an immunoglobulin molecule, a disulfide linked Fv, a monoclonal antibody, an affinity matured, a scFv, a chimeric antibody, a single domain antibody, a CDR-grafted antibody, a diabody, a humanized antibody, a multispecific antibody, a Fab, a dual specific antibody, a DVD, a Fab′, a bispecific antibody, a F(ab′)2, and a Fv.

In the method, the diagnosis may be confirmed by at least one of a lung biopsy, a magnetic resonance image (MRI), a CT scan, a positron emission tomography (PET) scan or combinations thereof.

The method may also comprise administering a lung cancer treatment regimen to the subject. The lung cancer treatment regimen in the method may comprise administering at least one of surgery, radiotherapeutic therapy, radiotherapeutic treatments, chemotherapy, targeted therapy or combinations thereof to the subject. The lung monitoring regimen may comprises at least one of conducting a chest computed tomography (CT) scan, a positron emission tomography (PET) scan, measuring lung function and determining MK, TFPI, NSE and CA19-9 levels at periodic intervals.

The present invention is also directed to a method for the diagnosis, prognosis and/or risk stratification of lung cancer in a subject having or suspected of lung cancer, the method comprising the step of detecting increased levels of midkine (MK), tissue factor pathway inhibitor (TFPI), neuron-specific enolase (NSE) and carbohydrate antigen 19-9 (CA19-9) and an increased amount of pack years of smoking by the subject relative to a control subject not having lung cancer.

The lung cancer identified pursuant to the above method may be early lung cancer. For example, the early lung cancer may be stage I lung cancer.

In the method, determining the level of MK, TFPI, NSE and CA19-9 may involve the step of contacting the biological sample with at least one antibody selected from the group consisting of: an antibody that specifically binds to MK, an antibody that specifically binds to TFPI, an antibody that specifically binds to NSE, an antibody that specifically binds to CA19-9 and combinations thereof.

In the method, determining the level of MK, TFPI, NSE and CA19-9 may involve the step of assaying the biological sample for MK, TFPI, NSE and CA19-9 by an immunoassay that employs at least one capture antibody and at least one antibody labeled with a detectable label, which generates a signal, and comprises comparing a signal generated by the detectable label as a direct or indirect indication of the amount of MK, TFPI, NSE and CA19-9 in the biological sample, wherein the capture antibody and the antibody labeled with a detectable label comprise:

a. at least one capture antibody that specifically binds to MK and at least one antibody labeled with a detectable label;

b. at least one capture antibody that specifically binds to TFPI and at least one antibody labeled with a detectable label;

c. at least one capture antibody that specifically binds to NSE and at least one antibody labeled with a detectable label; and

d. at least one capture antibody that specifically binds to CA19-9 and at least one antibody labeled with a detectable label.

The immunological method used in the method may comprise:
(a) measuring the levels of MK by:
   (i) contacting the test sample with at least one capture antibody, wherein the capture antibody binds to an epitope on MK or a fragment of MK to form a capture antibody-MK antigen complex;
   (ii) contacting the capture antibody-MK antigen complex with at least one detection antibody comprising a detectable label, wherein the detection antibody binds to an epitope on MK that is not bound by the capture antibody and forms a capture antibody-MK antigen-detection antibody complex; and
   (iii) determining the MK levels in the test sample based on the signal generated by the detectable label in the capture antibody-MK-9 antigen-detection antibody complex formed in (a)(ii);
(b) measuring the levels of TFPI by:
   (i) contacting the test sample with at least one capture antibody, wherein the capture antibody binds to an epitope on TFPI or a fragment of TFPI to form a capture antibody-TFPI antigen complex;
   (ii) contacting the capture antibody-TFPI antigen complex with at least one detection antibody comprising a detectable label, wherein the detection antibody binds to an epitope on TFPI that is not bound by the capture antibody and forms a capture antibody-TFPI antigen-detection antibody complex; and
   (iii) determining the TFPI levels in the test sample based on the signal generated by the detectable label in the capture antibody-TFPI antigen-detection antibody complex formed in (b)(ii);
(c) measuring the levels of NSE by:
   (i) contacting the test sample with at least one capture antibody, wherein the capture antibody binds to an epitope on NSE or a fragment of NSE to form a capture antibody-NSE antigen complex;
   (ii) contacting the capture antibody-NSE antigen complex with at least one detection antibody comprising a detectable label, wherein the detection antibody binds to an epitope on NSE that is not bound by the capture antibody and forms a capture antibody-NSE antigen-detection antibody complex; and
   (iii) determining the NSE levels in the test sample based on the signal generated by the detectable label in the capture antibody-NSE antigen-detection antibody complex formed in (c)(ii); and
(d) measuring the levels of CA19-9 by:
   (i) contacting the test sample with at least one capture antibody, wherein the capture antibody binds to an epitope on CA19-9 or a fragment of CA19-9 to form a capture antibody-CA19-9 antigen complex;
   (ii) contacting the capture antibody-CA19-9 antigen complex with at least one detection antibody comprising a detectable label, wherein the detection antibody binds to an epitope on CA19-9 that is not bound by the capture antibody and forms a capture antibody-CA19-9 antigen-detection antibody complex; and
   (iii) determining the CA19-9 levels in the test sample based on the signal generated by the detectable label in the capture antibody-CA19-9 antigen-detection antibody complex formed in (d)(ii).

In the method, the antibody may be selected from the group consisting of: a polyclonal antibody, a monoclonal antibody, a human antibody, an immunoglobulin molecule, a disulfide linked Fv, a monoclonal antibody, an affinity matured, a scFv, a chimeric antibody, a single domain antibody, a CDR-grafted antibody, a diabody, a humanized antibody, a multispecific antibody, a Fab, a dual specific antibody, a DVD, a Fab′, a bispecific antibody, a F(ab′)2, and a Fv.

In the method, the diagnosis may be confirmed by at least one of a lung biopsy, a magnetic resonance image (MRI), a CT scan, a positron emission tomography (PET) scan or combinations thereof.

The method may also comprise administering a lung cancer treatment regimen to the subject. The lung cancer treatment regimen in the method may comprise administering at least one of surgery, radiotherapeutic therapy, radiotherapeutic treatments, chemotherapy, targeted therapy or combinations thereof to the subject. The lung monitoring regimen may comprises at least one of conducting a chest computed tomography (CT) scan, a positron emission tomography (PET) scan, measuring lung function and determining MK, TFPI, NSE and CA19-9 levels at periodic intervals.

The present invention is also directed to a method of determining whether a subject is suffering from early lung cancer or benign lung disease, the method comprising the steps of: obtaining a biological sample from a subject; determining the levels of midkine (MK), tissue factor pathway inhibitor (TFPI), neuron-specific enolase (NSE) and carbohydrate antigen 19-9 (CA19-9) in the biological sample from the subject; determining the number of pack years of smoking of the subject; comparing the levels of MK, TFPI, NSE and CA19-9 in the biological sample to reference levels of MK, TFPI, NSE and CA19-9; comparing the number of pack years of smoking by the subject to a reference level of pack years; and providing a diagnosis of a subject having lung cancer if the levels of in the MK, TFPI, NSE and CA19-9 in the biological sample are greater than the reference levels of MK, TFPI, NSE and CA19-9 and the number of pack years of the subject are greater than the reference level of pack years or a diagnosis of a subject having benign disease if the levels of MK, TFPI, NSE and CA19-9 in the biological sample are equal or less than the reference levels of MK, TFPI, NSE and CA19-9 and the number of pack years of the subject are equal or less than the reference level of pack years.

The early lung cancer identified pursuant to the above method may Stage I or Stage II non-small cell lung cancer or limited stage small cell lung cancer. The reference levels of MK, TFPI, NSE and CA19-9 may be the MK, TFPI, NSE and CA19-9 cutoff values determined by a receiver operating curve (ROC) analysis from biological samples of a patient group. Alternatively, the reference levels of MK, TFPI, NSE and CA19-9 are the MK, TFPI, NSE and CA19-9 cutoff values may be determined by a quartile analysis of biological samples of a patient group. The MK, TFPI, NSE and CA19-9 reference level may be higher than or equal to 0.05 ng/mL, 0.06 ng/mL, 0.07 ng/mL, 0.08 ng/mL, 0.09 ng/mL, 0.10 ng/mL, 0.20 ng/mL, 0.30 ng/mL or 0.40 ng/mL in serum for MK in combination with levels higher than or equal 20 pg/mL, 21 pg/mL, 22 pg/mL, 23 pg/mL, 24 pg/mL, 25 pg/mL, 26 pg/mL, 27 pg/mL, 28 pg/mL, 29 pg/mL, 30 pg/mL, 31 pg/mL, 32 pg/mL, 33 pg/mL, 34 pg/mL, 35 pg/mL, 36 pg/mL, 37 pg/mL, 38 pg/mL, 39 pg/mL or 40 pg/mL in serum for TFPI, levels higher than or equal to 1 ng/mL, 2 ng/mL, 3 ng/mL, 4 ng/mL or 5 ng/mL in serum for NSE and levels higher than or equal to 10 U/mL, 11 U/mL, 12 U/mL, 13 U/mL, 14 U/mL, 15 U/mL, 16 U/mL, 17 U/mL, 18 U/mL, 19 U/mL, 30 U/mL, 31 U/mL, 32 U/mL, 33 U/mL, 34 U/mL, 35 U/mL, 36 U/mL or 37 U/mL in serum for CA19-9.

The method may comprise determining the level of at least one additional biomarker of lung cancer in the biological sample and comparing the level of the at least one additional biomarker of lung cancer to a reference level for the at least one biomarker of lung cancer. Additional biomarkers that may be determined may be selected from the group consisting of: nectin-4, cytokeratin 19-fragment 21-1 (CYFRA 21-1), carcinoembryonic antigen (CEA), progastrin releasing peptide (proGRP), carbohydrate antigen 125 (CA125), tissue polypeptide specific antigen (TPS), cancer antigen 15-3 (CA15-3), squamous cell carcinoma antigen (CCA), Helicobacter pylori, parainfluenza and combinations thereof.

The subject in the method may be a human. The biological sample obtained from the subject may be selected from a tissue sample, bodily fluid, whole blood, plasma, serum, urine, bronchoalveolar lavage fluid, and a cell culture suspension or fraction thereof. For example, the biological sample of the subject may be a blood plasma or blood serum.

The levels of MK, TFPI, NSE and CA19-9 can be determined by an immunological method using one or more molecules that specifically binding to MK, TFPI, NSE or CA19-9. For example, a molecule that specifically binds to MK, TFPI, NSE or CA19-9 is at least one antibody that is capable of specifically binding MK, TFPI, NSE or CA19-9.

In the method, if the levels of MK, TFPI, NSE or CA19-9 are above the cutoff values, this indicates the subject is suffering from lung cancer. In the method, if the number of pack years is above the reference level, this indicates that the subject is suffering from lung cancer.

The lung cancer treatment regimen in the method may comprise administering at least one of surgery, radiotherapeutic therapy, radiotherapeutic treatments, chemotherapy, targeted therapy or combinations thereof to the subject. The lung monitoring regimen may comprises at least one of conducting a chest computed tomography (CT) scan, a positron emission tomography (PET) scan, measuring lung function and determining MK, TFPI, NSE and CA19-9 levels at periodic intervals.

In the method, determining the level of MK, TFPI, NSE and CA19-9 may involve the step of contacting the biological sample with at least one antibody selected from the group consisting of: an antibody that specifically binds to MK, an antibody that specifically binds to TFPI, an antibody that specifically binds to NSE, an antibody that specifically binds to CA19-9 and combinations thereof.

In the method, determining the level of MK, TFPI, NSE and CA19-9 may involve the step of assaying the biological sample for MK, TFPI, NSE and CA19-9 by an immunoassay that employs at least one capture antibody and at least one antibody labeled with a detectable label, which generates a signal, and comprises comparing a signal generated by the detectable label as a direct or indirect indication of the amount of MK, TFPI, NSE and CA19-9 in the biological sample, wherein the capture antibody and the antibody labeled with a detectable label comprise:

a. at least one capture antibody that specifically binds to MK and at least one antibody labeled with a detectable label;

b. at least one capture antibody that specifically binds to TFPI and at least one antibody labeled with a detectable label;

c. at least one capture antibody that specifically binds to NSE and at least one antibody labeled with a detectable label; and

d. at least one capture antibody that specifically binds to CA19-9 and at least one antibody labeled with a detectable label.

The immunological method used in the method may comprise:
(a) measuring the levels of MK by:
   (i) contacting the test sample with at least one capture antibody, wherein the capture antibody binds to an epitope on MK or a fragment of MK to form a capture antibody-MK antigen complex;
   (ii) contacting the capture antibody-MK antigen complex with at least one detection antibody comprising a detectable label, wherein the detection antibody binds to an epitope on MK that is not bound by the capture antibody and forms a capture antibody-MK antigen-detection antibody complex; and
   (iii) determining the MK levels in the test sample based on the signal generated by the detectable label in the capture antibody-MK-9 antigen-detection antibody complex formed in (a)(ii);
(b) measuring the levels of TFPI by:
   (i) contacting the test sample with at least one capture antibody, wherein the capture antibody binds to an epitope on TFPI or a fragment of TFPI to form a capture antibody-TFPI antigen complex;
   (ii) contacting the capture antibody-TFPI antigen complex with at least one detection antibody comprising a detectable label, wherein the detection antibody binds to an epitope on TFPI that is not bound by the capture antibody and forms a capture antibody-TFPI antigen-detection antibody complex; and
   (iii) determining the TFPI levels in the test sample based on the signal generated by the detectable label in the capture antibody-TFPI antigen-detection antibody complex formed in (b)(ii);
(c) measuring the levels of NSE by:
   (i) contacting the test sample with at least one capture antibody, wherein the capture antibody binds to an epitope on NSE or a fragment of NSE to form a capture antibody-NSE antigen complex;
   (ii) contacting the capture antibody-NSE antigen complex with at least one detection antibody comprising a detectable label, wherein the detection antibody binds to an epitope on NSE that is not bound by the capture antibody and forms a capture antibody-NSE antigen-detection antibody complex; and
   (iii) determining the NSE levels in the test sample based on the signal generated by the detectable label in the capture antibody-NSE antigen-detection antibody complex formed in (c)(ii); and
(d) measuring the levels of CA19-9 by:
   (i) contacting the test sample with at least one capture antibody, wherein the capture antibody binds to an epitope on CA19-9 or a fragment of CA19-9 to form a capture antibody-CA19-9 antigen complex;
   (ii) contacting the capture antibody-CA19-9 antigen complex with at least one detection antibody comprising a detectable label, wherein the detection antibody binds to an epitope on CA19-9 that is not bound by the capture antibody and forms a capture antibody-CA19-9 antigen-detection antibody complex; and
   (iii) determining the CA19-9 levels in the test sample based on the signal generated by the detectable label in the capture antibody-CA19-9 antigen-detection antibody complex formed in (d)(ii).

In the method, the antibody may be selected from the group consisting of: a polyclonal antibody, a monoclonal antibody, a human antibody, an immunoglobulin molecule, a disulfide linked Fv, a monoclonal antibody, an affinity matured, a scFv, a chimeric antibody, a single domain antibody, a CDR-grafted antibody, a diabody, a humanized antibody, a multispecific antibody, a Fab, a dual specific antibody, a DVD, a Fab′, a bispecific antibody, a F(ab′)2, and a Fv.

In the method, the diagnosis may be confirmed by at least one of a lung biopsy, a magnetic resonance image (MRI), a CT scan, a positron emission tomography (PET) scan or combinations thereof.

The present invention is also directed to a kit for performing the above described methods, the kit may comprise: at least one reagent capable of specifically binding MK, TFPI, NSE and CA19-9 to quantify the levels of MK, TFPI, NSE and CA19-9 in the biological sample of a subject; a reference standard indicating reference levels of MK, TFPI, NSE and CA19-9; and a reference standard indicating a reference level of pack years. This kit may also contain a set of instructions for use thereof.

The kit may contain a reagent capable of specifically binding to MK, a reagent capable of specifically binding to TFPI, a reagent capable of specifically binding to NSE and a reagent capable of specifically binding to CA19-9 to quantify the levels of MK, TFPI, NSE and CA19-9 in the biological sample of a subject. For example, the kit may contain at least one reagent that comprises an antibody capable of specifically binding MK, at least one reagent that comprises an antibody capable of specifically binding TFPI, at least one reagent that comprises an antibody capable of specifically binding NSE and at least one reagent that comprises an antibody capable of specifically binding CA19-9. The kit may further contain one or more antibodies that are labeled with a detectable label. For example, the kit may contain one antibody labeled with a detectable label, two antibodies labeled with a detectable label, three antibodies labeled with a detectable label, four antibodies labeled with a detectable label, five antibodies labeled with a detectable label, six antibodies labeled with a detectable label, etc.

The kit may comprise at least one additional reagent capable of binding at least one additional biomarker of nectin-4, cytokeratin 19-fragment 21-1 (CYFRA 21-1), carcinoembryonic antigen (CEA), progastrin releasing peptide (proGRP), carbohydrate antigen 125 (CA125), tissue polypeptide specific antigen (TPS), cancer antigen 15-3 (CA15-3), squamous cell carcinoma antigen (CCA), Helicobacter pylori, and/or parainfluenza in the biological sample to quantify the concentration of the at least one additional biomarker in the biological sample, and a reference standard indicating a reference level of the at least one additional biomarker of nectin-4, cytokeratin 19-fragment 21-1 (CYFRA 21-1), carcinoembryonic antigen (CEA), progastrin releasing peptide (proGRP), carbohydrate antigen 125 (CA125), tissue polypeptide specific antigen (TPS), cancer antigen 15-3 (CA15-3), squamous cell carcinoma antigen (CCA), Helicobacter pylori and/or parainfluenza in the biological sample.

### BRIEF DESCRIPTION OF THE DRAWINGS

FIG. 1 shows blood levels of midkine (MK) in normal patients, patients suffering from benign lung disease, patients suffering from early lung cancer (stage I and stage II) and patients suffering from late stage lung cancer (stage III and stage IV). Boxes show medians and IQRs, whiskers shown 5^{th} and 95^{th} percentiles. “+” means >1.5 to <3 inter quartile range (IQR). The “x” with vertical line through middle means >3 IQR.

FIG. 2 shows blood levels of tissue factor pathway inhibitor (TFPI) in normal patients, patients suffering from benign lung disease, patients suffering from early lung cancer (stage I and stage II) and patients suffering from late stage lung cancer (stage III and stage IV). Boxes show medians and IQRs, whiskers shown 5^{th} and 95^{th} percentiles. “+” means >1.5 to <3 inter quartile range (IQR). The “x” with vertical line through middle means >3 IQR.

FIG. 3 shows blood levels of neuron-specific enolase (NSE) in normal patients, patients suffering from benign lung disease, patients suffering from early lung cancer (stage I and stage II) and patients suffering from late stage lung cancer (stage III and stage IV). Boxes show medians and IQRs, whiskers shown 5^{th} and 95^{th} percentiles. “+” means >1.5 to <3 inter quartile range (IQR). The “x” with vertical line through middle means >3 IQR.

FIG. 4 shows blood levels of carbohydrate antigen 19-9 (CA19-9) in normal patients, patients suffering from benign lung disease, patients suffering from early lung cancer (stage I and stage II) and patients suffering from late stage lung cancer (stage III and stage IV). Boxes show medians and IQRs, whiskers shown 5^{th} and 95^{th} percentiles. “+” means >1.5 to <3 inter quartile range (IQR). The “x” with vertical line through middle means >3 IQR.

FIG. 5 shows the raw p-value and the p-value after adjustment using the positive FDR (pFDR) method for the markers TFP1, LMNK, MK, Nectin-4, CA124, CA15-3, CA19-9, CEA, CYFRA21, H. pylori, NSE, parainfluenza, SCC, TPS, proGRP, pack years and age as described in Example 2.

FIG. 6 shows a graphical display of the exploratory principal component analysis performed for the markers TFP1, LMNK, MK, Nectin-4, CA124, CA15-3, CA19-9, CEA, CYFRA21, H. pylori, NSE, parainfluenza, SCC, TPS, proGRP, pack years and age as described in Example 2.

FIG. 7 shows the hierarchical clustering analysis of the markers TFPI, LNMK, H. pylori, pack years, CA19-9, CA15-3, CA125, SCC, Nectin-4, CEA, CYFRA21-1, TPS, parainfluenza, age, NSE and proGRP as described in Example 2.

FIG. 8 shows a plot of the linear discriminant analysis for the markers pack years, LNMK, TFP1 and CA19-9 as described in Example 2.

FIG. 9 shows the results of partition analysis performed to identify markers that might be associated with early lung cancer as described in Example 2. Persons with more pack years of smoking are more likely to have early cancer (boxes with hash marks) and those with lower values are more likely to be benign (gray boxes with no hash marks).

FIG. 10 shows the ARCHITECT® sandwich immunoassay used in the study described in Example 1.

FIG. 11 is a schematic of the enzyme-linked immunoassays (ELISA) used in the study described in Example 1. In this figure: (1) Plate is coated with a capture antibody; (2) sample is added, and any antigen (protein biomarker for pancreatic cancer) present binds to capture antibody; (3) detecting antibody is added, and binds to antigen; (4) enzyme-linked secondary antibody is added, and binds to detecting antibody; and (5) substrate is added, and is converted by enzyme to a detectable form.

### DETAILED DESCRIPTION

The present invention is directed to analyzing the levels of midkine (MK), tissue factor pathway inhibitor (TFPI), neuron-specific enolase (NSE) and carbohydrate antigen 19-9 (CA19-9) and determining the number of pack years of smoking of a subject to identify, diagnose and treat lung cancer in subject in need thereof. The biomarkers and number of pack years of smoking of the patient can be used to identify or diagnosis a patient with lung cancer, particularly, early lung cancer, to identify whether a patient is suffering from lung cancer or benign lung disease, to identify a patient as a candidate for a lung cancer treatment regimen, to classify a patient's risk of developing lung cancer, as well as to determining a diagnosis, prognosis or treatment regimen. The methods described herein can be adapted for use in an automated system or a semi-automated system. The methods of the present invention differ over previous lung cancer diagnostic methods by using a unique combination of (1) MK, TFPI, NSE and CA19-9 biomarkers and (2) the number of pack years of smoking of a subject to identify and diagnose patients suffering from lung cancer, particularly early lung cancer (or early lung cancer).

Section headings as used in this section and the entire disclosure herein are merely for organization purposes and are not intended to be limiting.

### A. DEFINITIONS

As used herein, the singular forms “a,” “an” and “the” include plural referents unless the context clearly dictates otherwise. For the recitation of numeric ranges herein, each intervening number there between with the same degree of precision is explicitly contemplated. For example, for the range 6-9, the numbers 7 and 8 are contemplated in addition to 6 and 9, and for the range 6.0-7.0, the numbers 6.0, 6.1, 6.2, 6.3, 6.4, 6.5, 6.6, 6.7, 6.8, 6.9 and 7.0 are explicitly contemplated.

The use of “or” means “and/or” unless stated otherwise. Furthermore, the use of the terms “including” and “having,” as well as other forms of those terms, such as “includes,” “included”, “has,” and “have” are not limiting.

The “area under curve” or “AUC” refers to area under a ROC curve. AUC under a ROC curve is a measure of accuracy. An area of 1 represents a perfect test, whereas an area of 0.5 represents an insignificant test. A preferred AUC may be at least approximately 0.700, at least approximately 0.750, at least approximately 0.800, at least approximately 0.850, at least approximately 0.900, at least approximately 0.910, at least approximately 0.920, at least approximately 0.930, at least approximately 0.940, at least approximately 0.950, at least approximately 0.960, at least approximately 0.970, at least approximately 0.980, at least approximately 0.990, or at least approximately 0.995.

“Benign lung disease” as used herein refers to diseases other than cancer that affect the lungs and breathing in a patient. Examples of benign lung disease include one or more of the following: chronic obstructive pulmonary disease (COPD), emphysema, bronchitis, acute inflammation, dysplasia, chronic lung inflammation, acute lung inflammation, benign neoplasia, sarcoidosis, pneumonia, bronchiectasis, benign pulmonary nodules, hemoptysis, atelectasis, asthma, benign lung tumors. Subjects with benign lung disease tend to be at higher risk of developing lung cancer.

“Cancer antigen 15-3” (CA15-3) refers to a member of the mucin family of glycoproteins that is typically elevated in breast cancer. CA15-3 is approved for monitoring of advanced breast cancer according to the NACB (National Academy of Clinical Biochemists) guidelines. For breast cancer, the ASCO guidelines recommend that CA15-3 not be used alone but be used with CEA and CA27.29 (Tietz Textbook of Clinical Chemistry and Molecular Diagnostics, 2012, Burtis, C., Ashwood, E., and D. Bruns, Elsevier Publishing). CA125 is FDA approved for monitoring response to therapy for patients with epithelial ovarian cancer. Serial testing should be used in conjunction with other clinical methods for monitoring ovarian cancer. Values of CA125 are defined by using the OC 125 monoclonal antibody. Serum CA125 values are useful for monitoring the course of disease in patients with invasive ovarian cancer (Abbott ARCHITECT CA125 II Package Insert 016-623 4/09).

“Carbohydrate antigen 19-9” (CA19-9) refers to a mucin-glycoprotein derived from a human colorectal carcinoma cell line. It is related to the Lewis blood group protein and is present in the epithelial tissue of the stomach, gall bladder, pancreas, and prostate. CA19-9 has been FDA approved for use as an aid in the management of pancreatic cancer and is intended to be used in conjunction with other diagnostic information such as CT and MRI imaging procedures. CA19-9 belongs to the sialylated Lewis blood group antigen. Some individuals may be undetectable for CA19-9 because they are Lewis antigen negative. Increased serum CA19-9 may also be elevated in patients with nonmalignant conditions such as pancreatitis and other gastrointestinal disorders (Abbott ARCHITECT CA19-9 XR Package Insert 015-550 11/05).

“Carbohydrate antigen 125” (CA125)” refers to a member of the mucin family of glycoproteins that is elevated in several different types of cancers. CA125 is approved as an aid in diagnosis and for monitoring therapy, detection of recurrence and prognosis (Tietz Textbook of Clinical Chemistry and Molecular Diagnostics, 2012, Burtis, C., Ashwood, E., and D. Bruns, Elsevier Publishing). CA125 is FDA approved for monitoring response to therapy for patients with epithelial ovarian cancer. Serial testing should be used in conjunction with other clinical methods for monitoring ovarian cancer. Values of CA125 are defined by using the OC 125 monoclonal antibody. Serum CA125 values are useful for monitoring the course of disease in patients with invasive ovarian cancer (Abbott ARCHITECT CA125 II Package Insert 016-623 4/09).

“Carcinoembryonic antigen” (CEA) refers to a glycosyl phosphatidyl inositol (GPI)-cell surface anchored glycoprotein whose specialized sialofucosylated glycoforms serve as functional colon carcinoma L-selectin and E-selectin ligands. CEA is involved in cell adhesion. CEA is a tumor associated antigen first described by Gold and Freedman in 1965 (J Exp Med 121; 439-462). CEA is characterized by a glycoprotein that is approximately 200 kDa in size. CEA is FDA approved and intended to be used as an aid in the prognosis and management of cancer patients in patients with changing concentrations of CEA. Clinical relevance has been shown in colorectal, gastric, lung, prostate, pancreatic, and ovarian cancers (Abbott ARCHITECT CEA Package Insert 34-4067/R4).

The “confidence interval” or “CI” as used herein refers to an interval estimate of a population parameter used to indicate the reliability of an estimate. The confidence interval refers to the region containing the limits or band of a parameter with an associated confidence level that the bounds are large enough to contain the true parameter value. The bands may be single-sided to describe an upper or lower limit or double sided to describe both upper and lower limits. The region gives a range of values, bounded below by a lower confidence limit and from above by an upper confidence limit, such that one can be confident (at a pre-specified level such as 95% or 99%) that the true population parameter value is included within the confidence interval. Confidence intervals may be formed for any of the parameters used to describe the characteristic of interest. Confidence intervals may be used to estimate the population parameters from the sample statistics and allow a probabilistic quantification of the strength of the best estimate. A preferred confidence interval may be at least 90%, at least 91%, at least 92%, at least 93%, at least 94%, at least 95%, at least 96%, at least 97%, at least 98%, or at least 99%.

“Correlation coefficient” as used herein means a significant correlation may be determined by any suitable statistic method. For example, the correlation coefficient may be Spearman's rank correlation coefficient (also known as “Spearman's rho” and “Spearman's rho correlation coefficient”), which is a non-parametric measure of statistical dependence between two variables. Spearman's rho assesses how well the relationship between two variables can be described using a monotonic function. If there are no repeated data values, a perfect Spearman correlation of +1 or −1 occurs when each of the variables is a perfect monotone function of the other. A highly significant correlation is indicated when Spearman's rho is at least 0.50, preferably at least 0.60, more preferably at least 0.70, even more preferably at least 0.80, yet more preferably at least 0.85, even more preferably at least 0.90. Spearman's rho may be between 0.55 and 0.60. Most preferably, for two markers, Spearman's rho is at least approximately 0.50, at least approximately 0.55, at least approximately 0.60, at least approximately 0.65, at least approximately 0.70, at least approximately 0.75, at least approximately 0.80, at least approximately 0.85, at least approximately 0.90, at least approximately 0.91, at least approximately 0.92, at least approximately 0.93, at least approximately 0.94, at least approximately 0.95, at least approximately 0.96, at least approximately 0.97, at least approximately 0.98, or at least approximately 0.99.

“Cytokeratins” are a group of approximately 20 proteins that make up the cytoskeletal intermediate filaments of epithelial cells and cells of epithelial origin. The cytokeratins can be divided into two main groups: type 1 is smaller and acidic and type 2 is larger and neutral to basic. Clinically useful members of the family include cytokeratin 19-fragment 21-1 (CYFRA 21-1). “CYFRA21-1” refers to a cytokeratin 19 fragment that is soluble in serum. Although expressed in all body fluids, its major occurrence is in the lung. CYFRA21-1 is elevated in all types of lung cancer and positively correlate with advancing stage and are useful in monitoring the disease course and provide postsurgical followup. (Tietz Textbook of Clinical Chemistry and Molecular Diagnostics, 2012, Burtis, C., Ashwood, E., and D. Bruns, Elsevier Publishing).

“Early lung cancer” or “early stage lung cancer” as used interchangeably herein means Stage I or Stage II (including Stage IIA or Stage IIB) lung cancer when referring to non-small cell lung cancer and limited stage small cell lung cancer when refer to small cell lung cancer.

“Interquartile range” or “IQR” as used herein means a measure of statistical dispersion, being equal to the difference between the upper and lower quartiles, IQR=Q3−Q1.

“Helicobacter pylori” (H. pylori) as used herein refers to a Gram-negative, microaerophilic bacterium found in the stomach that can cause digestive illnesses, including gastritis and peptic ulcer disease. Most estimates suggest that the organism is present in the mucous layer of the stomach in half of the population of the world. In Europe, 30-50% of adults and in the United States, at least 20% of the adult population, are infected with H. pylori. (Tietz Textbook of Clinical Chemistry and Molecular Diagnostics, 2012, Burtis, C., Ashwood, E., and D. Bruns, Elsevier Publishing.).

“Lung cancer” refers to the uncontrolled growth of abnormal cells that start off or begin in one or both lungs in a subject. Different types of lung cancer include non-small cell lung cancer (NSCLC) and small cell lung cancer (SCLC). Non-small cell lung cancer is the most common form of lung cancer accounting for 80% of all lung cancers.

Non-small cell lung cancers are assigned a stage from I to IV in order of severity. “Stage I” lung cancer is confined to one or both of the lungs and has not spread to any lymph nodes. “Stage II” lung cancer has spread from one or both of the lungs to one or more local lymph nodes. If the patient is found to have one or more pulmonary nodules that are less than 3 cm and the cancer has spread to one or more local lymph nodes, then the cancer is considered to be “Stage IIA”. If the patient is found to have a one or more pulmonary nodules that are greater than 3 cm and the cancer has spread to one or more local lymph nodes, then the cancer is considered to be “Stage IIB”. “Stage III” lung cancer has spread from one or both of the lungs to one or more lymph nodes in the middle of the chest. If the cancer has spread only to lymph nodes on the same side of the chest where the cancer started, then the cancer is considered to be “Stage IIIA”. If the cancer has spread to the lymph nodes on the opposite side of the chest, or above the collar bone, then the cancer is considered to be “Stage IIIB”. “Stage IV” lung cancer is the most advanced lung cancer and has spread from one or both of the lungs to fluid in the area around the lungs, or to another part of the body, such as the liver of other organs.

Small cell lung cancers are staged using a two-tiered system. Limited stage SCLC refers to cancer that is confined to one or both lungs and lymph nodes. In extensive stage SCLC, the cancer has spread beyond one or both of the lungs to other parts of the body.

“Midkine” (MK) as used herein means a heparin-binding growth factor that is the product of a retinoic acid-responsive gene. Midkine is a secreted protein that has been found to be overexpressed in various human malignancies, such as bladder, prostate, breast, lung, liver, and colon tumors, whereas its expression is typically low or undetectable in normal adult tissues. MK is involved in various cellular processes such as cellular proliferation, survival, and migration. In addition to these typical growth factor activities, MK exhibits several other activities related to fibrinolysis, blood pressure, host defense and other processes. Many cell-surface receptors have been identified to account for the multiple biological activities of MK. The expression of MK is frequently upregulated in many types of human carcinoma. Moreover, blood MK levels are closely correlated with patient outcome. Knockdown and blockade of MK suppress tumorigenesis and tumor development. Thus, MK serves as a tumor marker and a molecular target for cancer therapy. Furthermore, there is growing evidence that MK plays pivotal roles in neural and inflammatory diseases. (Sakamoto K and Kadomatsu K. Pathol Int. 2012 July; 62(7):445-55).

“Nectin-4” as used herein refers to a 66-kDa adhesion molecule of the Nectin family. Nectins are a family of immunoglobulin-like cell adhesion molecules important in the formation and maintenance of adherens junctions and tight junctions. All nectins share a similar structure: 3 immunoglobulin-like extracellular loops, a single transmembrane region, and a short cytoplasmic domain that can bind to afadin. Nectins function by first forming homo cis-dimers on the cell surface and then trans-dimers on adjacent cells in a homophilic and a heterophilic manner. The specificity of binding is different for each nectin. For example, nectin-4 can form trans-homodimers and trans-heterodimers with nectin 1 but not with nectin 2 or 3. Four nectin proteins have been described. Nectin-1 and 2 are broadly expressed in adult tissues, while nectin-3 is expressed mainly in the testes and placenta. Expression of nectin-4 is normally restricted to the placenta but has been reported in ductal breast carcinoma and lung adenocarcinomas (DeRycke, M. S. et al., Am J Clin Pathol. 2010 November; 134(5): 835-845).

“Neuron-specific enolase” (NSE) as used herein refers to the most abundant form of the glycolytic enolase found in adult neurons. Of the three enolase subunits (α, β, and γ), NSE is a dimer composed of two γ subunits. The enzyme is found in neuronal tissue and cells of the diffuse neuroendocrine system. NSE therefore is associated with tumors of neuroendocrine origin. NSE is released into the blood as a result of cell lysis as opposed to secretion. (Tietz Textbook of Clinical Chemistry and Molecular Diagnostics, 2012, Burtis, C., Ashwood, E., and D. Bruns, Elsevier Publishing).

“Pack years”, pack years of smoking” or “pack years smoked” as used herein refers to the number of years a subject has smoked multiplied by the average number of packs smoked per day. A person who has smoked, on average, 1 pack of cigarettes per day for 35 years is referred to have 35 pack years of smoking history.

“Progastrin releasing peptide” (proGRP) as used herein refers to a member of the bombesin family of peptides. Progastrin releasing peptide has been shown to have mitogenic activity in small cell lung carcinoma (SCLC), and to be produced by SCLC in an autocrine fashion. ProGRP (residues 31-98) is a precursor of a neuropeptide hormone named gastrin-releasing peptide (GRP), and is frequently produced by small cell lung cancer (SCLC) cells. Circulating proGRP levels have been shown to serve as a reliable marker in SCLC patients. ProGRP has been reported to be the most sensitive marker for discriminating SCLC from benign diseases of the lung. If measured with serum neuron-specific enolase at the same time, proGRP is known to provide additive information on pathological characteristics of lung cancer. Moreover, proGRP was found to be useful in the monitoring of response to therapy and for the

“Parainfluenza” as used herein refers to group of viruses that lead to upper and lower respiratory infections. Symptoms vary depending on the type of infection. Parainfluenza viruses (PIV) are common respiratory viruses that belong to the Paramyxoviridae family and include four serotypes and two subtypes (1, 2, 3, 4a, and 4b). PIV infection leads to a wide variety of clinical syndromes ranging from mild upper respiratory illness (URI) to severe pneumonia (A. R. Falsey, Infect Drug Resist. 2012; 5: 121-127).

“Predetermined cutoff” “cutoff”, “predetermined level” and “reference level” as used herein refers to an assay cutoff value that is used to assess diagnostic, prognostic, or therapeutic efficacy results by comparing the assay results against the predetermined cutoff/level, where the predetermined cutoff/level already has been linked or associated with various clinical parameters (e.g., presence of disease, stage of disease, severity of disease, progression, non-progression, or improvement of disease, etc.). The disclosure provides exemplary predetermined levels and reference levels. However, it is well-known that cutoff values may vary depending on the nature of the immunoassay (e.g., antibodies employed, reaction conditions, sample purity, etc.). It further is well within the ordinary skill of one in the art to adapt the disclosure herein for other immunoassays to obtain immunoassay-specific cutoff values for those other immunoassays based on the description provided by this disclosure. Whereas the precise value of the predetermined cutoff/level may vary between assays, the correlations as described herein should be generally applicable.

“Risk assessment,” “risk classification,” “risk identification,” or “risk stratification” of subjects (e.g., patients) as used herein refers to the evaluation of factors including biomarkers, to predict the risk of occurrence of future events including disease onset or disease progression, so that treatment decisions regarding the subject may be made on a more informed basis.

“Sample,”, “biological sample”, “test sample,” “specimen,” “sample from a subject,” and “patient sample” as used herein may be used interchangeable and may be a sample of blood, tissue, urine, serum, plasma, amniotic fluid, cerebrospinal fluid, placental cells or tissue, endothelial cells, leukocytes, or monocytes. The sample can be used directly as obtained from a patient or can be pre-treated, such as by filtration, distillation, extraction, concentration, centrifugation, inactivation of interfering components, addition of reagents, and the like, to modify the character of the sample in some manner as discussed herein or otherwise as is known in the art.

Any cell type, tissue, or bodily fluid may be utilized to obtain a sample. Such cell types, tissues, and fluid may include sections of tissues such as biopsy and autopsy samples, frozen sections taken for histologic purposes, blood (such as whole blood), plasma, serum, sputum, stool, tears, mucus, saliva, bronchoalveolar lavage (BAL) fluid, hair, skin, red blood cells, platelets, interstitial fluid, ocular lens fluid, cerebral spinal fluid, sweat, nasal fluid, synovial fluid, menses, amniotic fluid, semen, etc. Cell types and tissues may also include lymph fluid, ascetic fluid, gynecological fluid, urine, peritoneal fluid, cerebrospinal fluid, a fluid collected by vaginal rinsing, or a fluid collected by vaginal flushing. A tissue or cell type may be provided by removing a sample of cells from an animal, but can also be accomplished by using previously isolated cells (e.g., isolated by another person, at another time, and/or for another purpose). Archival tissues, such as those having treatment or outcome history, may also be used. Protein or nucleotide isolation and/or purification may not be necessary.

“Squamous cell carcinoma antigen” (CCA) as used herein refers to a protein that belongs to the serine protease inhibitor (Serpin) family of proteins. Elevated expression of SCCA has been used as a biomarker for aggressive squamous cell carcinoma (SCC) in cancers of the cervix, lung, head and neck, liver, skin, digestive tract, ovaries and urogenital tract. SCC antigen concentrations may be elevated in certain benign conditions, including pulmonary infection, skin disease, renal failure and liver disease. (Tietz Textbook of Clinical Chemistry and Molecular Diagnostics, 2012, Burtis, C., Ashwood, E., and D. Bruns, Elsevier Publishing).

“Subject” and “patient” as used herein interchangeably refers to any vertebrate, including, but not limited to, a mammal (e.g., cow, pig, camel, llama, horse, goat, rabbit, sheep, hamsters, guinea pig, cat, dog, rat, and mouse, a non-human primate (for example, a monkey, such as a cynomolgous or rhesus monkey, chimpanzee, etc) and a human). In some embodiments, the subject may be a human or a non-human. The subject or patient may be undergoing other forms of treatment.

“Tissue factor pathway inhibitor” (TFPI) as used herein refers to a protease inhibitor that regulates the tissue factor (TF)-dependent pathway of blood coagulation. The coagulation process initiates with the formation of a factor VIIa-TF complex, which proteolytically activates additional proteases (factors IX and X) and ultimately leads to the formation of a fibrin clot. (Tietz Textbook of Clinical Chemistry and Molecular Diagnostics, 2012, Burtis, C., Ashwood, E., and D. Bruns, Elsevier Publishing). The full-length TFPI is a 40 kDa glycoprotein with 304 amino acids. It consists of an N-terminal acidic region, three Kunitz domains, and a C-terminal basic domain. In circulation, about 10% of TFPI is carried by platelets. Among the rest, the majority circulates as bound forms (Novotny et al., 1991, Blood 78, 394).

“Tissue polypeptide specific antigens” (TPS) as used herein refers to the M3 epitope of tissue polypeptide antigen that is elevated in several different types of cancers. TPS is actually the antigenic site on cytokeratin 18 that is recognized as the M3 monoclonal antibody, hence the M3 epitope. This epitope has been proposed as a specific marker of cell proliferation and is detectable in serum. (Tietz Textbook of Clinical Chemistry and Molecular Diagnostics, 2012, Burtis, C., Ashwood, E., and D. Bruns, Elsevier Publishing).

The terms “treat,” “treated,” or “treating” as used herein refers to a therapeutic wherein the object is to slow down (lessen) an undesired physiological condition, disorder or disease, or to obtain beneficial or desired clinical results. For the purposes of this invention, beneficial or desired clinical results include, but are not limited to, alleviation of symptoms; diminishment of the extent of the condition, disorder or disease; stabilization (i.e., not worsening) of the state of the condition, disorder or disease; delay in onset or slowing of the progression of the condition, disorder or disease; amelioration of the condition, disorder or disease state; and remission (whether partial or total), whether detectable or undetectable, or enhancement or improvement of the condition, disorder or disease. Treatment also includes prolonging survival as compared to expected survival if not receiving treatment.

Unless otherwise defined herein, scientific and technical terms used in connection with the present disclosure shall have the meanings that are commonly understood by those of ordinary skill in the art. For example, any nomenclatures used in connection with, and techniques of, cell and tissue culture, molecular biology, immunology, microbiology, genetics and protein and nucleic acid chemistry and hybridization described herein are those that are well known and commonly used in the art. The meaning and scope of the terms should be clear; in the event however of any latent ambiguity, definitions provided herein take precedent over any dictionary or extrinsic definition. Further, unless otherwise required by context, singular terms shall include pluralities and plural terms shall include the singular.

### B. METHODS OF IDENTIFYING OR DETERMINING SUBJECTS HAVING OR AT RISK OF DEVELOPING LUNG CANCER

The present invention is directed to identifying a subject having or at risk of developing lung cancer by (1) quantifying or determining the levels of the following combination of markers: midkine (MK), tissue factor pathway inhibitor (TFPI), neuron-specific enolase (NSE) and carbohydrate antigen 19-9 (CA19-9) and (2) determining the number of pack years of smoking of the subject. Generally, a subject is a high risk individual, i.e., a patient who has a family history of lung history, who has a history of smoking or currently is a smoker, who has been diagnosed as having benign lung disease, who has actually been diagnosed as having or being at risk for lung cancer and/or who demonstrates unfavorable concentrations or amounts of MK, TFPI, NSE and CA19-9, as described herein. Specifically, such a method can comprise the steps of: of: (1) obtaining a biological sample from a subject; (2) determining the levels of midkine (MK), tissue factor pathway inhibitor (TFPI), neuron-specific enolase (NSE) and carbohydrate antigen 19-9 (CA19-9) in the biological sample from the subject; (3) determining the number of pack years of smoking of the subject; (4) comparing the levels of MK, TFPI, NSE and CA19-9 in the biological sample to reference levels of MK, TFPI, NSE and CA19-9; (5) comparing the number of pack years of smoking by the subject to a reference level of pack years; and (6) determining or identifying the subject as having lung cancer or at risk for lung cancer if the levels of MK, TFPI, NSE and CA19-9 in the biological sample are greater or unfavorable with respect to the reference levels of MK, TFPI, NSE and CA19-9 and the number of pack years of smoking by the subject is greater than or unfavorable with respect to the reference level of pack years. The subject is determined or identified not to have or be at risk for lung cancer if the levels of MK, TFPI, NSE and CA19-9 in the biological sample are less than or favorable with respect to the reference levels of MK, TFPI, NSE and CA19-9 and the number of pack years of smoking by the subject is less than or favorable with respect to the reference level of pack years.

The methods described herein can be used to identify a subject having or at risk of having or developing early lung cancer, such as Stage I or Stage II when referring to NSCLC or limited stage small cell lung cancer when referring to small cell lung cancer.

The method may further comprise confirming a diagnosis or risk of lung cancer (particularly early lung cancer). The method may still further comprise the step of a treatment and/or monitoring regimen.

### C. METHODS OF PROVIDING A DIAGNOSIS OF LUNG CANCER

The present invention is also directed to providing a diagnosis of lung cancer by (1) quantifying or determining the levels of the following combination of markers: midkine (MK), tissue factor pathway inhibitor (TFPI), neuron-specific enolase (NSE) and carbohydrate antigen 19-9 (CA19-9) and (2) determining the number of pack years of smoking of the subject.

The method includes the steps of: (1) obtaining a biological sample from a subject; (2) determining the levels of midkine (MK), tissue factor pathway inhibitor (TFPI), neuron-specific enolase (NSE) and carbohydrate antigen 19-9 (CA19-9) in the biological sample from the subject; (3) determining the number of pack years of smoking of the subject; (4) comparing the levels of MK, TFPI, NSE and CA19-9 in the biological sample to reference levels of MK, TFPI, NSE and CA19-9; (5) comparing the number of pack years of smoking by the subject to a reference level of pack years; and (6) providing a diagnosis to a subject of having lung cancer if the levels of MK, TFPI, NSE and CA19-9 in the biological sample are greater than or unfavorable with respect to the reference levels of MK, TFPI, NSE and CA19-9 and the number of pack years of smoking by the subject is greater than or unfavorable with respect to the reference level of pack years. A subject is determined not to have lung cancer if cancer if the levels of MK, TFPI, NSE and CA19-9 in the biological sample are less than or favorable with respect to the reference levels of MK, TFPI, NSE and CA19-9 and the number of pack years of smoking by the subject is less than or favorable with respect to the reference level of pack years.

The methods described herein can be used to provide a diagnosis of early lung cancer, such as Stage I or Stage II when referring to NSCLC or limited stage small cell lung cancer when referring to small cell lung cancer.

The method may further comprise confirming a diagnosis or risk of lung cancer. The method may still further comprise the step of a treatment and/or monitoring regimen.

### D. METHODS OF PROVIDING A DIAGNOSIS OF EARLY LUNG CANCER

The present invention is also directed to providing a diagnosis of early lung cancer by (1) quantifying or determining the levels of the following combination of markers: midkine (MK), tissue factor pathway inhibitor (TFPI), neuron-specific enolase (NSE) and carbohydrate antigen 19-9 (CA19-9) and (2) determining the number of pack years of smoking of the subject.

The method includes the steps of: (1) obtaining a biological sample from a subject; (2) determining the levels of midkine (MK), tissue factor pathway inhibitor (TFPI), neuron-specific enolase (NSE) and carbohydrate antigen 19-9 (CA19-9) in the biological sample from the subject; (3) determining the number of pack years of smoking of the subject; (4) comparing the levels of MK, TFPI, NSE and CA19-9 in the biological sample to reference levels of MK, TFPI, NSE and CA19-9; (5) comparing the number of pack years of smoking by the subject to a reference level of pack years; and (6) providing a diagnosis to a subject of having early lung cancer if the levels of MK, TFPI, NSE and CA19-9 in the biological sample are greater than or unfavorable with respect to the reference levels of MK, TFPI, NSE and CA19-9 and the number of pack years of smoking by the subject is greater than or unfavorable with respect to the reference level of pack years. A subject is determined not to have early lung cancer if cancer if the levels of MK, TFPI, NSE and CA19-9 in the biological sample are less than or favorable with respect to the reference levels of MK, TFPI, NSE and CA19-9 and the number of pack years of smoking by the subject is less than or favorable with respect to the reference level of pack years.

The methods described herein can be used to provide a diagnosis of Stage I or Stage II when referring to NSCLC or limited stage small cell lung cancer when referring to small cell lung cancer.

The method may further comprise confirming a diagnosis or risk of lung cancer. The method may still further comprise the step of a treatment and/or monitoring regimen.

### E. METHOD FOR DIAGNOSIS, PROGNOSIS, AND/OR RISK STRATIFICATION OF LUNG CANCER

The present invention is also directed to providing a diagnosis, prognosis, and/or risk stratification of lung cancer, particularly, early lung cancer, in a subject having or suspected of having lung cancer by (1) quantifying or determining the levels of the biomarkers midkine (MK), tissue factor pathway inhibitor (TFPI), neuron-specific enolase (NSE) and carbohydrate antigen 19-9 (CA19-9) in a biological sample from the subject and (2) determining the number of pack years of smoking of the subject. By measuring the levels of midkine (MK), tissue factor pathway inhibitor (TFPI), neuron-specific enolase (NSE) and carbohydrate antigen 19-9 (CA19-9), the method allows the early detection of lung cancer to be more accurately diagnosed and subsequently treated more successfully, compared to other commercially available assays.

The method includes the steps of obtaining a biological sample from a subject, determining the level of MK, TFPI, NSE and CA19-9 in the biological sample, determining the number of pack years of smoking of the subject; comparing the levels of MK, TFPI, NSE and CA19-9 in the biological sample to levels of the respective combination of markers (i.e., MK, TFPI, NSE and CA19-9 in the biological sample is compared to a reference level of MK, TFPI, NSE and CA19-9) in a subject having benign lung disease and comparing the number of pack years of the subject with a reference level of pack years of smoking in a subject having benign lung disease and providing a diagnosis, prognosis and/or risk stratification of lung cancer in the subject if the levels of MK, TFPI, NSE and CA19-9 in the biological sample are greater or unfavorable with respect to the reference levels of MK, TFPI, NSE and CA19-9 and the number of pack years of smoking by the subject is greater or unfavorable than the reference level of pack years. The method may further comprise confirming a diagnosis or risk of lung cancer (particularly early lung cancer). The method may still further comprise the step of a treatment and/or monitoring regimen.

### F. METHODS OF DIFFERENTIATING BETWEEN SUBJECTS WITH EARLY LUNG CANCER AND BENIGN LUNG DISEASE

The present invention is also directed to determining (or diagnosing) or differentiating whether a subject has or is suffering from early lung cancer or benign lung disease by (1) quantifying or determining the levels of the following combination of markers: midkine (MK), tissue factor pathway inhibitor (TFPI), neuron-specific enolase (NSE) and carbohydrate antigen 19-9 (CA19-9) and (2) determining the number of pack years of smoking of the subject.

The method includes the steps of: (1) obtaining a biological sample from a subject; (2) determining the levels of midkine (MK), tissue factor pathway inhibitor (TFPI), neuron-specific enolase (NSE) and carbohydrate antigen 19-9 (CA19-9) in the biological sample from the subject; (3) determining the number of pack years of smoking of the subject; (4) comparing the levels of MK, TFPI, NSE and CA19-9 in the biological sample to reference levels of MK, TFPI, NSE and CA19-9; (5) comparing the number of pack years of smoking by the subject to a reference level of pack years; and (6) providing a (i) diagnosis of a subject having early lung cancer if the levels of in the MK, TFPI, NSE and CA19-9 in the biological sample are greater than or unfavorable with respect to the reference levels of MK, TFPI, NSE and CA19-9 and the number of pack years of the subject are greater than or unfavorable with respect to the reference level of pack years or a (ii) diagnosis of a subject having benign disease if the levels of MK, TFPI, NSE and CA19-9 in the biological sample are equal or less than or favorable with respect to the reference levels of MK, TFPI, NSE and CA19-9 and the number of pack years of the subject are equal or less than or favorable with respect to the reference level of pack years.

The method described herein can be used to identify whether a subject may have Stage I or Stage II NSCLC.

The method may further comprise confirming a diagnosis or risk of early lung cancer (particularly early lung cancer). The method may still further comprise the step of a treatment and/or monitoring regimen.

### G. METHODS OF MONITORING THE PROGRESSION OF LUNG CANCER IN A SUBJECT

The methods described herein also can be used to monitor the progression of lung cancer, particularly early lung cancer, in a subject by determining the levels of MK, TFPI, NSE and CA19-9 in a subject. Optimally, the method includes the steps of (a) determining the concentrations or amounts of MK, TFPI, NSE and CA19-9 in a test sample from a subject, (b) determining the concentrations or amounts of MK, TFPI, NSE and CA19-9 in a later test sample from a subject, and (c) comparing the concentrations or amounts of MK, TFPI, NSE and CA19-9 as determined in step (b) with the concentrations or amounts of MK, TFPI, NSE and CA19-9 determined in step (a), wherein if the concentrations or amounts determined in step (b) is greater than, unchanged or is unfavorable when compared to the concentrations or amounts of MK, TFPI, NSE and CA19-9 determined in step (a), then the disease in the subject is determined to have continued, progressed or worsened. By comparison, if the concentrations or amounts of MK, TFPI, NSE and CA19-9 as determined in step (b) is lower or favorable when compared to the concentrations or amounts of MK, TFPI, NSE and CA19-9 as determined in step (a), then the lung cancer in the subject is determined to have discontinued, regressed or improved.

Optionally, the method further comprises comparing the concentrations or amounts of MK, TFPI, NSE and CA19-9 as determined in step (b), for example, with predetermined levels. Further, optionally the method comprises treating the subject with one or more treatment regimens (namely, surgery, chemotherapy, radiotherapeutic therapy, radiotherapeutic treatments, target therapy or any combination thereof) for a period of time if the comparison shows that the concentrations or amounts of MK, TFPI, NSE and CA19-9 as determined in step (b), for example, are greater than or unfavorably altered with respect to the predetermined levels.

Still further, the methods can be used to monitor a subject receiving treatment with one or more of surgery, chemotherapy, radiotherapeutic therapy, radiotherapeutic treatments, target therapy or any combination thereof (collectively “treatment regimens”). Specifically, such methods involve providing a first test sample from a subject before the subject has been administered one or more treatment regimens. Next, the concentrations or amounts in a first test sample from a subject of MK, TFPI, NSE and CA19-9 are determined (e.g., using methods known in the art). After the concentrations or amounts of MK, TFPI, NSE and CA19-9 are determined, optionally the concentrations or amounts of MK, TFPI, NSE and CA19-9 are then compared with predetermined levels. If the concentrations or amounts of MK, TFPI, NSE and CA19-9 as determined in the first test sample are lower or favorably with respect to the predetermined levels, then the subject is not treated with one or more treatment regimens or, alternatively, the subject may be treated with one or more treatment regimens. If the concentrations or amounts of MK, TFPI, NSE and CA19-9 as determined in the first test sample are higher or unfavorably with respect to the predetermined levels, then the subject is treated with one or more treatment regimens for a period of time or alternatively, the subject is not treated with one or more treatment regimens. The period of time that the subject is treated with the one or more treatment regimens can be determined by one skilled in the art (for example, the period of time can be from about seven (7) days to about two years, preferably from about fourteen (14) days to about one (1) year).

During the course of treatment with the one or more treatment regimens, second and subsequent test samples are then obtained from the subject. The number of test samples and the time in which said test samples are obtained from the subject are not critical. For example, a second test sample could be obtained seven (7) days after the subject is first administered the one or more treatment regimens, a third test sample could be obtained two (2) weeks after the subject is first administered the one or more treatment regimens, a fourth test sample could be obtained three (3) weeks after the subject is first administered the one or more treatment regimens, a fifth test sample could be obtained four (4) weeks after the subject is first administered the one or more treatment regimens, etc.

After each second or subsequent test sample is obtained from the subject, the concentrations or amounts MK, TFPI, NSE and CA19-9 are determined in the second or subsequent test sample (e.g., using methods known in the art). The concentrations or amounts of MK, TFPI, NSE and CA19-9 as determined in each of the second and subsequent test samples are then compared with the concentrations or amounts of MK, TFPI, NSE and CA19-9 as determined in the first test sample (e.g., the test sample that was originally optionally compared to the predetermined level). If the concentrations or amounts of MK, TFPI, NSE and CA19-9 as determined in step (c) are lower than or favorable when compared to the concentrations or amounts of MK, TFPI, NSE and CA19-9 as determined in step (a), then the disease in the subject is determined to have discontinued, regressed, or improved, and the subject should continue to be administered the one or treatment regimens of step (b). However, if the concentrations or amounts determined in step (c) are greater than, unchanged or are unfavorable when compared to the concentrations or amounts of MK, TFPI, NSE and CA19-9 as determined in step (a), then the disease in the subject is determined to have continued, progressed or worsened, and the subject should be treated with a higher concentration of the one or more treatment regimens administered to the subject in step (b) or the subject should be treated with one or more treatment regimens that are different from the one or more treatment regimens administered to the subject in step (b). Specifically, the subject can be treated with one or more treatment regimens that are different from the one or more treatment regimens that the subject had previously received to decrease or lower said subject's MK, TFPI, NSE and CA19-9 levels.

Furthermore, the above assays can be performed using a first test sample obtained from a subject where the first test sample is obtained from one source, such as urine, serum, or plasma. Optionally the above assays can then be repeated using a second test sample obtained from the subject where the second test sample is obtained from another source. For example, if the first test sample was obtained from urine, the second test sample can be obtained from serum or plasma. The results obtained from the assays using the first test sample and the second test sample can be compared. The comparison can be used to assess the status of a disease or condition in the subject.

### H. REFERENCE LEVELS

The methods described herein use reference levels of MK, TFPI, NSE and CA19-9 and pack years of smoking of a subject to (1) determine whether or not a subject has or is at risk of having or developing lung cancer in a subject; (2) provide a diagnosis of lung cancer in a subject; (3) provide a diagnosis of early lung cancer in a subject; (4) provide a diagnosis, prognosis and/or risk stratification of lung cancer in a subject having or suspected of lung cancer; (5) determine or diagnose whether or not a subject has early lung cancer or benign lung disease; and (6) monitor the progression of lung cancer in a subject. Levels higher than or equal to the reference levels of MK, TFPI, NSE and CA19-9 and pack years of smoking identify the patient as having lung cancer or at risk of having or developing lung cancer or early lung cancer. Levels lower than the reference level of MP, TFPI, NSE and CA19-9 and pack years of smoking identify the patient as having benign lung disease.

### i. Reference Levels for Markers MK, TFPI, NSE and CA19-9

Generally, predetermined or reference levels can be employed as a benchmark against which to assess results obtained upon assaying a test sample for MK, TFPI, NSE and CA19-9. Generally, in making such a comparison, the predetermined levels are obtained by running a particular assay a sufficient number of times and under appropriate conditions such that a linkage or association of the analyte present, amount or concentration with a particular stage or endpoint of lung cancer with particular indicia can be made. Typically, the predetermined levels are obtained with assays of reference subjects (or populations of subjects). The MK, TFPI, NSE and CA19-9 measured can include MK, TFPI, NSE and CA19-9 fragments thereof, degradation products thereof, and/or enzymatic cleavage products thereof.

In particular, with respect to predetermined levels as employed for monitoring disease progression and/or treatment, the amount or concentration of MK, TFPI, NSE and CA19-9 or MK, TFPI, NSE and CA19-9 fragments may be “unchanged,” “favorable” (or “favorably altered”), or “unfavorable” (or “unfavorably altered”). “Elevated” or “increased” refers to an amount or a concentration in a test sample that is higher or greater than a typical or normal level or range (e.g., predetermined level), or is higher or greater than another reference level or range (e.g., earlier or baseline sample). The term “lowered” or “reduced” refers to an amount or a concentration in a test sample that is lower or less than a typical or normal level or range (e.g., predetermined level), or is lower or less than another reference level or range (e.g., earlier or baseline sample). The term “altered” refers to an amount or a concentration in a sample that is altered (increased or decreased) over a typical or normal level or range (e.g., predetermined level), or over another reference level or range (e.g., earlier or baseline sample).

The typical or normal levels or ranges for MK, TFPI, NSE and CA19-9 are defined in accordance with standard practice. A so-called altered level or alteration can be considered to have occurred when there is any net change as compared to the typical or normal level or range, or reference level or range that cannot be explained by experimental error or sample variation. Thus, the level measured in a particular sample will be compared with the level or range of levels determined in similar samples from a so-called normal subject. In this context, a “normal” (sometimes termed “control” or “healthy”) subject is an individual with no detectable benign lung disease or cancer, and a “normal” patient or population is/are one(s) that exhibit(s) no detectable benign lung disease or cancer, respectively, for example. An “apparently normal subject” is one in which MK, TFPI, NSE and CA19-9 has not been or is being assessed. The level of an analyte is said to be “elevated” when the analyte is normally undetectable (e.g., the normal level is zero, or within a range of from about 25 to about 75 percentiles of normal populations), but is detected in a test sample, as well as when the analyte is present in the test sample at a higher than normal level.

For example, in one aspect, reference levels higher (or greater) than or equal to 0.05 ng/mL, 0.06 ng/mL, 0.07 ng/mL, 0.08 ng/mL, 0.09 ng/mL, 0.10 ng/mL, 0.20 ng/mL, 0.30 ng/mL or 0.40 ng/mL in serum for MK in combination with levels higher than or equal 20 pg/mL, 21 pg/mL, 22 pg/mL, 23 pg/mL, 24 pg/mL, 25 pg/mL, 26 pg/mL, 27 pg/mL, 28 pg/mL, 29 pg/mL, 30 pg/mL, 31 pg/mL, 32 pg/mL, 33 pg/mL, 34 pg/mL, 35 pg/mL, 36 pg/mL, 37 pg/mL, 38 pg/mL, 39 pg/mL or 40 pg/mL in serum for TFPI, levels higher than or equal to 1 ng/mL, 2 ng/mL, 3 ng/mL, 4 ng/mL or 5 ng/mL in serum for NSE and levels higher than or equal to 10 U/mL, 11 U/mL, 12 U/mL, 13 U/mL, 14 U/mL, 15 U/mL, 16 U/mL, 17 U/mL, 18 U/mL, 19 U/mL, 30 U/mL, 31 U/mL, 32 U/mL, 33 U/mL, 34 U/mL, 35 U/mL, 36 U/mL or 37 U/mL in serum for CA19-9 identify the subject as having lung cancer. In another aspect, reference levels higher (or greater) than or equal to 0.05 to 0.4 ng/mL in serum for MK, in combination with levels higher than or equal to 20 to 40 pg/mL in serum for TFPI, levels higher than or equal to 1 to 5 ng/mL in serum for NSE and levels higher than or equal to 10 to 37 U/mL in serum for CA19-9 identify the subject as having lung cancer.

Cutoff values (or predetermined cutoff values) may be determined by a receiver operating curve (ROC) analysis from biological samples of the patient group. ROC analysis, as generally known in the biological arts, is a determination of the ability of a test to discriminate one condition from another, e.g., to determine the performance of each marker in identifying a patient having lung cancer. A description of ROC analysis as applied according to the present disclosure is provided in P. J. Heagerty et al., Time-dependent ROC curves for censored survival data and a diagnostric marker, Biometrics 56:337-44 (2000), the disclosure of which is hereby incorporated by reference in its entirety. Alternatively, cutoff values can be determined by a quartile analysis of biological samples of a patient group. For example, a cutoff value can be determined by selecting a value that corresponds to any value in the 25th-75th percentile range, preferably a value that corresponds to the 25th percentile, the 50th percentile or the 75th percentile, and more preferably the 75th percentile.

Such statistical analyses can be performed using any method known in the art and can be implemented through any number of commercially available software packages (e.g., from Analyse-it Software Ltd., Leeds, UK; StataCorp LP, College Station, Tex.; SAS Institute Inc., Cary, N.C.).

### ii. Reference Levels for Pack Years of Smoking

The methods use reference levels of pack years of smoking to determine whether or not a subject has or is at risk of having or developing lung cancer. The reference level of pack years of smoking may be a predetermined cutoff value or the number of pack years of smoking determined from a control subject that benign lung disease. The reference level for pack years of smoking is greater or equal to thirty (30) pack years of smoking and less than fifteen (15) years since quitting smoking (See, Tammeagi M C, et al., N. Engl. J. Med. 2013 Feb. 21, 368(8):728-36).

Cutoff values (or predetermined cutoff values) may be determined by a receiver operating curve (ROC) analysis from biological samples of the patient group. ROC analysis, as generally known in the biological arts, is a determination of the ability of a test to discriminate one condition from another, e.g., to determine the performance of each marker in identifying a patient having lung cancer. A description of ROC analysis as applied according to the present disclosure is provided in P. J. Heagerty et al., Time-dependent ROC curves for censored survival data and a diagnostric marker, Biometrics 56:337-44 (2000), the disclosure of which is hereby incorporated by reference in its entirety. Alternatively, cutoff values can be determined by a quartile analysis of biological samples of a patient group. For example, a cutoff value can be determined by selecting a value that corresponds to any value in the 25th-75th percentile range, preferably a value that corresponds to the 25th percentile, the 50th percentile or the 75th percentile, and more preferably the 75th percentile.

Such statistical analyses can be performed using any method known in the art and can be implemented through any number of commercially available software packages (e.g., from Analyse-it Software Ltd., Leeds, UK; StataCorp LP, College Station, Tex.; SAS Institute Inc., Cary, N.C.).

### I. ADDITIONAL BIOMARKERS

The methods as described herein can further comprise quantifying five or more biomarkers in combination with the specific biomarker combinations discussed above (i.e., MK, TFPI, NSE and CA19-9). The method may further comprising quantifying or determining the level of at least one additional biomarker of lung cancer in the biological sample and comparing the level of the at least one additional biomarker of lung cancer to a reference level for the at least one biomarker of lung cancer (such as early lung cancer) in the biological sample. The additional biomarkers can be nectin-4, cytokeratin 19-fragment 21-1 (CYFRA 21-1), carcinoembryonic antigen (CEA), progastrin releasing peptide (proGRP), carbohydrate antigen 125 (CA125), tissue polypeptide specific antigen (TPS), cancer antigen 15-3 (CA15-3), squamous cell carcinoma antigen (CCA), Helicobacter pylori, parainfluenza or any combinations thereof. Levels higher than or equal to the reference level of at least one of nectin-4, cytokeratin 19-fragment 21-1 (CYFRA 21-1), carcinoembryonic antigen (CEA), progastrin releasing peptide (proGRP), carbohydrate antigen 125 (CA125), tissue polypeptide specific antigen (TPS), cancer antigen 15-3 (CA15-3), squamous cell carcinoma antigen (CCA), Helicobacter pylori, parainfluenza and combinations thereof, can be used in the methods of the present invention to (1) determine whether or not a subject has or is at risk of having or developing lung cancer in a subject; (2) provide a diagnosis of lung cancer in a subject; (3) provide a diagnosis of early lung cancer in a subject; (4) provide a diagnosis, prognosis and/or risk stratification of lung cancer in a subject having or suspected of lung cancer; (5) determine or diagnose whether or not a subject has early lung cancer or benign lung disease; and (6) monitor the progression of lung cancer in a subject.

### i. Reference Levels for Additional Biomarkers

Generally, predetermined or reference levels can be employed as a benchmark against which to assess results obtained upon assaying a test sample for nectin-4, CYFRA 21-1, CEA, proGRP, CA125, TPS, CA15-3, CCA, Helicobacter pylori, parainfluenza or any combinations thereof. Generally, in making such a comparison, the predetermined levels are obtained by running a particular assay a sufficient number of times and under appropriate conditions such that a linkage or association of the analyte present, amount or concentration with a particular stage or endpoint of lung cancer with particular indicia can be made. Typically, the predetermined levels are obtained with assays of reference subjects (or populations of subjects). The nectin-4, CYFRA 21-1, CEA, proGRP, CA125, TPS, CA15-3, CCA, Helicobacter pylori, parainfluenza measured can include nectin-4, CYFRA 21-1, CEA, proGRP, CA125, TPS, CA15-3, CCA, Helicobacter pylori, parainfluenza fragments thereof, degradation products thereof, and/or enzymatic cleavage products thereof.

In particular, with respect to predetermined levels as employed for monitoring disease progression and/or treatment, the amount or concentration of nectin-4, CYFRA 21-1, CEA, proGRP, CA125, TPS, CA15-3, CCA, Helicobacter pylori, parainfluenza or nectin-4, CYFRA 21-1, CEA, proGRP, CA125, TPS, CA15-3, CCA, Helicobacter pylori, parainfluenza fragments may be “unchanged,” “favorable” (or “favorably altered”), or “unfavorable” (or “unfavorably altered”). “Elevated” or “increased” refers to an amount or a concentration in a test sample that is higher or greater than a typical or normal level or range (e.g., predetermined level), or is higher or greater than another reference level or range (e.g., earlier or baseline sample). The term “lowered” or “reduced” refers to an amount or a concentration in a test sample that is lower or less than a typical or normal level or range (e.g., predetermined level), or is lower or less than another reference level or range (e.g., earlier or baseline sample). The term “altered” refers to an amount or a concentration in a sample that is altered (increased or decreased) over a typical or normal level or range (e.g., predetermined level), or over another reference level or range (e.g., earlier or baseline sample).

The typical or normal levels or ranges for nectin-4, CYFRA 21-1, CEA, proGRP, CA125, TPS, CA15-3, CCA, Helicobacter pylori, parainfluenza are defined in accordance with standard practice. A so-called altered level or alteration can be considered to have occurred when there is any net change as compared to the typical or normal level or range, or reference level or range that cannot be explained by experimental error or sample variation. Thus, the level measured in a particular sample will be compared with the level or range of levels determined in similar samples from a so-called normal subject. In this context, a “normal” (sometimes termed “control” or “healthy”) subject is an individual with no detectable benign lung disease or cancer, and a “normal” patient or population is/are one(s) that exhibit(s) no detectable benign lung disease or cancer, respectively, for example. An “apparently normal subject” is one in which nectin-4, CYFRA 21-1, CEA, proGRP, CA125, TPS, CA15-3, CCA, Helicobacter pylori, parainfluenza has not been or is being assessed. The level of an analyte is said to be “elevated” when the analyte is normally undetectable (e.g., the normal level is zero, or within a range of from about 25 to about 75 percentiles of normal populations), but is detected in a test sample, as well as when the analyte is present in the test sample at a higher than normal level.

Cutoff values (or predetermined cutoff values) may be determined by a receiver operating curve (ROC) analysis from biological samples of the patient group. ROC analysis, as generally known in the biological arts, is a determination of the ability of a test to discriminate one condition from another, e.g., to determine the performance of each marker in identifying a patient having lung cancer. A description of ROC analysis as applied according to the present disclosure is provided in P. J. Heagerty et al., Time-dependent ROC curves for censored survival data and a diagnostric marker, Biometrics 56:337-44 (2000), the disclosure of which is hereby incorporated by reference in its entirety.

Alternatively, cutoff values can be determined by a quartile analysis of biological samples of a patient group. For example, a cutoff value can be determined by selecting a value that corresponds to any value in the 25th-75th percentile range, preferably a value that corresponds to the 25th percentile, the 50th percentile or the 75th percentile, and more preferably the 75th percentile.

Such statistical analyses can be performed using any method known in the art and can be implemented through any number of commercially available software packages (e.g., from Analyse-it Software Ltd., Leeds, UK; StataCorp LP, College Station, Tex.; SAS Institute Inc., Cary, N.C.).

### J. MEANS FOR CONFIRMING THE LUNG CANCER STATUS OF A SUBJECT

The subject identified in the methods described above having levels of MK, TFPI, NSE and CA19-9 and pack years of smoking greater than the values discussed above are identified as patients suffering from lung cancer. The lung cancer can be early stage lung cancer. The subject may then be administered a means for confirming the lung cancer status. A means for confirming lung cancer status may include performing one or more of a lung biopsy of a pulmonary nodule or other mass, a magnetic resonance image (MRI), a chest tomography (CT) scan, a positron emission tomography (PET) scan or combinations thereof. The stage of the lung cancer may be confirmed by taking a biopsy of the pulmonary nodule or other mass.

### K. MEANS FOR MONITORING THE LUNG CANCER STATUS OF A SUBJECT

The subject identified in the methods described above having levels of MK, TFPI, NSE and CA19-9 and pack years of smoking greater than the values discussed are identified as patients suffering from lung cancer. The lung cancer can be early stage lung cancer. The subject may then be placed on a lung monitoring regimen. Specifically, the subject may be administered a means for monitoring the effectiveness of any treatment regimens (such as surgery, radiotherapeutic therapy, radiotherapeutic treatments, chemotherapy, target therapy, or any combinations thereof) being used to treat the cancer as well as to assess the progress (or lack thereof) of the disease. The lung monitoring regimen may involve conducting a chest tomography (CT) scan, a positron emission tomography (PET) scan, measuring lung function, determining MK, TFPI, NSE and CA19-9 at periodic intervals (such periodic intervals being once a week, once a month, once every two months, once every three months, once every four months, once every five months, once every six months, once every seven months, once every eight months, once every nine months, once every ten months, once every eleven months, and once a year) or any combinations thereof.

### L. TREATMENT REGIMENS

The subject identified in the methods described above having levels of MK, TFPI, NSE and CA19-9 and pack years of smoking greater than the values discussed are identified as patients suffering from lung cancer. The lung cancer can be early stage lung cancer. The subjects are then treated for lung cancer. Any number or variety of treatment regimens can be used. For example, such treatment regimens may include one or more of surgery, chemotherapy, radiotherapeutic therapy, radiotherapeutic treatments, target therapy or any combinations thereof.

Surgery may include a mediastinoscopy, thoracoscopy, wedge resection, segmentectomy, lobectomy, sleeve reaction, a pneumonectomy or combinations thereof.

Chemotherapy involves the use of one or more chemotherapeutic agents. The phrase “chemotherapeutic agent,” as used herein, is intended to refer to any chemotherapeutic agent known to those of skill in the art to be effective for the treatment or amelioration of cancer. Chemotherapeutic agents include, but are not limited to; small molecules; synthetic drugs; peptides; polypeptides; proteins; nucleic acids (e.g., DNA and RNA nucleotides including, but not limited to, antisense nucleotide sequences, triple helices and nucleotide sequences encoding biologically active proteins, polypeptides or peptides); antibodies; synthetic or natural inorganic molecules; mimetic agents; and synthetic or natural organic molecules. Any agent which is known to be useful, or which has been used or is currently being used for the treatment or amelioration of cancer can be used in combination with an active vitamin D compound in accordance with the invention described herein. See, e.g., Hardman et al., eds., 1996, Goodman & Gilman's The Pharmacological Basis Of Basis Of Therapeutics 9th Ed, Mc-Graw-Hill, New York, N.Y. for information regarding therapeutic agents which have been or are currently being used for the treatment or amelioration of cancer.

Chemotherapeutic agents may include alkylating agents, antimetabolites, anti-mitotic agents, epipodophyllotoxins, antibiotics, hormones and hormone antagonists, enzymes, platinum coordination complexes, anthracenediones, substituted ureas, methylhydrazine derivatives, imidazotetrazine derivatives, cytoprotective agents, DNA topoisomerase inhibitors, biological response modifiers, retinoids, therapeutic antibodies, differentiating agents, immunomodulatory agents, and angiogenesis inhibitors.

Examples of chemotherapeutic agents that may be used include those that have been used, are currently used, or are known to be useful for the treatment or amelioration of lung cancer. Preferred agents include, but are not limited to, cisplatin, carboplatin, paclitaxel, docetaxel, etoposide, vincristine, vinblastine, cyclophosphamide, doxorubicin, vinorelbine, topotecan, gemcitabine, irinotecan, gifitinib, ifosfamide, tarceva, oblimersen, and TLK286.

Other chemotherapeutic agents that may be used include abarelix, aldesleukin, alemtuzumab, alitretinoin, allopurinol, altretamine, amifostine, anastrozole, arsenic trioxide, asparaginase, azacytidine, BCG live, bevaceizumab, bexarotene, bleomycin, bortezomib, busulfan, calusterone, camptothecin, capecitabine, carboplatin, carmustine, celecoxib, cetuximab, chlorambucil, cinacalcet, cisplatin, cladribine, cyclophosphamide, cytarabine, dacarbazine, dactinomycin, darbepoetin alfa, daunorubicin, denileukin diftitox, dexrazoxane, docetaxel, doxorubicin, dromostanolone, Elliott's B solution, epirubicin, epoetin alfa, estramustine, etoposide, exemestane, filgrastim, floxuridine, fludarabine, fluorouracil, fulvestrant, gemcitabine, gemtuzumab ozogamicin, gefitinib, goserelin, hydroxyurea, ibritumomab tiuxetan, idarubicin, ifosfamide, imatinib, interferon alfa-2a, interferon alfa-2b, irinotecan, letrozole, leucovorin, levamisole, lomustine, meclorethamine, megestrol, melphalan, mercaptopurine, mesna, methotrexate, methoxsalen, methylprednisolone, mitomycin C, mitotane, mitoxantrone, nandrolone, nofetumomab, oblimersen, oprelvekin, oxaliplatin, paclitaxel, pamidronate, pegademase, pegaspargase, pegfilgrastim, pemetrexed, pentostatin, pipobroman, plicamycin, polifeprosan, porfimer, procarbazine, quinacrine, rasburicase, rituximab, sargramostim, SN-38, streptozocin, talc, tamoxifen, tarceva, temozolomide, teniposide, testolactone, thioguanine, thiotepa, topotecan, toremifene, tositumomab, trastuzumab, tretinoin, uracil mustard, valrubicin, vinblastine, vincristine, vinorelbine, and zoledronate.

Chemotherapeutic agents also include anti-inflammatory drugs which are known to be useful for ameliorating inflammation. Suitable anti-inflammatory drugs include, but are not limited to, salicylates (such as aspirin, choline inagnessium trisalicylate, methyl salicylate, salsalte and diflunisal), acetic acids (such as indomethacin, sulindac, tolmetin, aceclofenac and diclofenac), 2-arylpropionic acids or profens (such as ibuprofen, ketoprofen, naproxen, fenoprofen, flurbiprofen and oxaprozin), N-arylanthranilic acids or fenamic acids (such as mefenamic acid, flufenamic acid, and meclofenamate), enolic acids or oxicams (such as piroxicam and meloxicam), cox inhibitors (such as celecoxib, rofecoxib (withdrawn from market), valdecoxib, parecoxib and etoricoxib), sulphonanilides such as nimesulide; naphthylalkanones (such as nabumetone), pyranocarboxylic acids (such as etodolac) and pyrroles (such as ketorolac).

Chemotherapeutic agents further include immunomodulatory agents. As used herein, the phrase “immunomodulatory agent” and variations thereof including, but not limited to, immunomodulatory agents, immunomodulants, immunomodulators or immunomodulatory drugs, refer to an agent that modulates a host's immune system. In particular, an immunomodulatory agent is an agent that alters the ability of a subject's immune system to respond to one or more foreign antigens. In one aspect, an immunomodulatory agent is an agent that shifts one aspect of a subject's immune response, e.g., the agent shifts the immune response from a Th1 to a Th2 response. In another aspect, an immunomodulatory agent is an agent that inhibits or reduces a subject's immune system (i.e., an immunosuppressant agent). In still certain other aspects, an immunomodulatory agent is an agent that activates or increases a subject's immune system (i.e., an immunostimulatory agent).

Immunomodulatory agents that may be used include small molecules, peptides, polypeptides, proteins, nucleic acids (e.g., DNA and RNA nucleotides including, but not limited to, antisense nucleotide sequences, triple helices and nucleotide sequences encoding biologically active proteins, polypeptides or peptides), antibodies, synthetic or natural inorganic molecules, mimetic agents, and synthetic or natural organic molecules. A particularly useful immunomodulatory agent is thalidomide.

Examples of immunosuppressant agents that may be used in the methods of the present invention include glucocorticoid receptor agonists (e.g., cortisone, dexamethasone, hydrocortisone, betamethasone), calcineurin inhibitors (e.g., macrolides such as tacrolimus and pimecrolimus), immunophilins (e.g., cyclosporin A) and mTOR inhibitors (e.g., sirolimus, marketed as RAPAMUNE® by Wyeth). Immunostimulant agents useful for the present invention include interferon and Zidovudine (AZT).

Chemotherapeutic agents may be administered at doses that are recognized by those of skill in the art to be effective for the treatment of lung cancer.

Radiotherapeutic therapy involves the use of one or more radiotherapeutic agents. The phrase “radiotherapeutic agent,” as used herein, is intended to refer to any radiotherapeutic agent known to one of skill in the art to be effective to treat or ameliorate cancer, without limitation. For instance, the radiotherapeutic agent can be an agent such as those administered in brachytherapy or radionuclide therapy.

Brachytherapy can be administered according to any schedule, dose, or method known to one of skill in the art to be effective in the treatment or amelioration of cancer, without limitation. In general, brachytherapy comprises insertion of radioactive sources into the body of a subject to be treated for cancer, preferably inside the tumor itself, such that the tumor is maximally exposed to the radioactive source, while preferably minimizing the exposure of healthy tissue. Representative radioisotopes that can be administered in brachytherapy include, but are not limited to, phosphorus 32, cobalt 60, palladium 103, ruthenium 106, iodine 125, cesium 137, iridium 192, xenon 133, radium 226, californium 252, or gold 198. Methods of administering and apparatuses and compositions useful for brachytherapy are described in Mazeron et al., Sem. Rad. One. 12:95-108 (2002) and U.S. Pat. Nos. 6,319,189, 6,179,766, 6,168,777, 6,149,889, and 5,611,767, each of which is incorporated herein by reference in its entirety.

Radionuclide therapy can be administered according to any schedule, dose, or method known to one of skill in the art to be effective in the treatment or amelioration of cancer, without limitation. In general, radionuclide therapy comprises systemic administration of a radioisotope that preferentially accumulates in or binds to the surface of cancerous cells. The preferential accumulation of the radionuclide can be mediated by a number of mechanisms, including, but not limited to, incorporation of the radionuclide into rapidly proliferating cells, specific accumulation of the radionuclide by the cancerous tissue without special targeting, or conjugation of the radionuclide to a biomolecule specific for a neoplasm.

Representative radioisotopes that can be administered in radionuclide therapy include, but are not limited to, phosphorus 32, yttrium 90, dysprosium 165, indium 111, strontium 89, samarium 153, rhenium 186, iodine 131, iodine 125, lutetium 177, and bismuth 213. While all of these radioisotopes may be linked to a biomolecule providing specificity of targeting, iodine 131, indium 111, phosphorus 32, samarium 153, and rhenium 186 may be administered systemically without such conjugation. One of skill in the art may select a specific biomolecule for use in targeting a particular neoplasm for radionuclide therapy based upon the cell-surface molecules present on that neoplasm. Examples of biomolecules providing specificity for particular cell are reviewed in an article by Thomas, Cancer Biother. Radiopharm. 17:71-82 (2002), which is incorporated herein by reference in its entirety. Furthermore, methods of administering and compositions useful for radionuclide therapy may be found in U.S. Pat. Nos. 6,426,400, 6,358,194, 5,766,571, and 5,563,250, each of which is incorporated herein by reference in its entirety.

“Radiotherapeutic treatment,” as used herein, is intended to refer to any radiotherapeutic treatment known to one of skill in the art to be effective to treat or ameliorate cancer, without limitation. For instance, the radiotherapeutic treatment can be external-beam radiation therapy, thermotherapy, radiosurgery, charged-particle radiotherapy, neutron radiotherapy, or photodynamic therapy.

External-beam radiation therapy can be administered according to any schedule, dose, or method known to one of skill in the art to be effective in the treatment or amelioration of cancer, without limitation. In general, external-beam radiation therapy comprises irradiating a defined volume within a subject with a high energy beam, thereby causing cell death within that volume. The irradiated volume preferably contains the entire cancer to be treated, and preferably contains as little healthy tissue as possible. Methods of administering and apparatuses and compositions useful for external-beam radiation therapy can be found in U.S. Pat. Nos. 6,449,336, 6,398,710, 6,393,096, 6,335,961, 6,307,914, 6,256,591, 6,245,005, 6,038,283, 6,001,054, 5,802,136, 5,596,619, and 5,528,652, each of which is incorporated herein by reference in its entirety.

Thermotherapy can be administered according to any schedule, dose, or method known to one of skill in the art to be effective in the treatment or amelioration of cancer, without limitation. In certain embodiments, the thermotherapy can be cryoablation therapy. In other embodiments, the thermotherapy can be hyperthermic therapy. In still other embodiments, the thermotherapy can be a therapy that elevates the temperature of the tumor higher than in hyperthermic therapy.

Cryoablation therapy involves freezing of a neoplastic mass, leading to deposition of intra- and extra-cellular ice crystals; disruption of cellular membranes, proteins, and organelles; and induction of a hyperosmotic environment, thereby causing cell death. Methods for and apparatuses useful in cryoablation therapy are described in Murphy et al., Sem. Urol. Oncol. 19:133-140 (2001) and U.S. Pat. Nos. 6,383,181, 6,383,180, 5,993,444, 5,654,279, 5,437,673, and 5,147,355, each of which is incorporated herein by reference in its entirety.

Hyperthermic therapy typically involves elevating the temperature of a neoplastic mass to a range from about 42° C. to about 44° C. The temperature of the cancer may be further elevated above this range; however, such temperatures can increase injury to surrounding healthy tissue while not causing increased cell death within the tumor to be treated. The tumor may be heated in hyperthermic therapy by any means known to one of skill in the art without limitation. For example, and not by way of limitation, the tumor may be heated by microwaves, high intensity focused ultrasound, ferromagnetic thermoseeds, localized current fields, infrared radiation, wet or dry radiofrequency ablation, laser photocoagulation, laser interstitial thermic therapy, and electrocautery. Microwaves and radiowaves can be generated by waveguide applicators, horn, spiral, current sheet, and compact applicators.

Other methods of and apparatuses and compositions for raising the temperature of a tumor are reviewed in an article by Wust et al., Lancet Oncol. 3:487-97 (2002), and described in U.S. Pat. Nos. 6,470,217, 6,379,347, 6,165,440, 6,163,726, 6,099,554, 6,009,351, 5,776,175, 5,707,401, 5,658,234, 5,620,479, 5,549,639, and 5,523,058, each of which is incorporated herein by reference in its entirety.

Radiosurgery can be administered according to any schedule, dose, or method known to one of skill in the art to be effective in the treatment or amelioration of cancer, without limitation. In general, radiosurgery comprises exposing a defined volume within a subject to a manually directed radioactive source, thereby causing cell death within that volume. The irradiated volume preferably contains the entire cancer to be treated, and preferably contains as little healthy tissue as possible. Typically, the tissue to be treated is first exposed using conventional surgical techniques, then the radioactive source is manually directed to that area by a surgeon. Alternatively, the radioactive source can be placed near the tissue to be irradiated using, for example, a laparoscope. Methods and apparatuses useful for radiosurgery are further described in Valentini et al., Eur. J. Surg. Oncol. 28:180-185 (2002) and in U.S. Pat. Nos. 6,421,416, 6,248,056, and 5,547,454, each of which is incorporated herein by reference in its entirety.

Charged-particle radiotherapy can be administered according to any schedule, dose, or method known to one of skill in the art to be effective in the treatment or amelioration of cancer, without limitation. In certain embodiments, the charged-particle radiotherapy can be proton beam radiotherapy. In other embodiments, the charged-particle radiotherapy can be helium ion radiotherapy. In general, charged-particle radiotherapy comprises irradiating a defined volume within a subject with a charged-particle beam, thereby causing cellular death within that volume. The irradiated volume preferably contains the entire cancer to be treated, and preferably contains as little healthy tissue as possible. A method for administering charged-particle radiotherapy is described in U.S. Pat. No. 5,668,371, which is incorporated herein by reference in its entirety.

Neutron radiotherapy can be administered according to any schedule, dose, or method known to one of skill in the art to be effective in the treatment or amelioration of cancer, without limitation. In certain embodiments, the neutron radiotherapy can be a neutron capture therapy. In such embodiments, a compound that emits radiation when bombarded with neutrons and preferentially accumulates in a neoplastic mass is administered to a subject. Subsequently, the tumor is irradiated with a low energy neutron beam, activating the compound and causing it to emit decay products that kill the cancerous cells. The compound to be activated can be caused to preferentially accumulate in the target tissue according to any of the methods useful for targeting of radionuclides, as described above, or in the methods described in Laramore, Semin. Oncol. 24:672-685 (1997) and in U.S. Pat. Nos. 6,400,796, 5,877,165, 5,872,107, and 5,653,957, each of which is incorporated. herein by reference in its entirety.

In other embodiments, the neutron radiotherapy can be a fast neutron radiotherapy. In general, fast neutron radiotherapy comprises irradiating a defined volume within a subject with a neutron beam, thereby causing cellular death within that volume.

Photodynamic therapy can be administered according to any schedule, dose, or method known to one of skill in the art to be effective in the treatment or amelioration of cancer, without limitation. In general, photodynamic therapy comprises administering a photosensitizing agent that preferentially accumulates in a neoplastic mass and sensitizes the neoplasm to light, then exposing the tumor to light of an appropriate wavelength. Upon such exposure, the photosensitizing agent catalyzes the production of a cytotoxic agent, such as, e.g., singlet oxygen, which kills the cancerous cells. Methods of administering and apparatuses and compositions useful for photodynamic therapy are disclosed in Hopper, Lancet Oncol. 1:212-219 (2000) and U.S. Pat. Nos. 6,283,957, 6,071,908, 6,011,563, 5,855,595, 5,716,595, and 5,707,401, each of which is incorporated herein by reference in its entirety.

Radiotherapy can be administered to destroy tumor cells before or after surgery, before or after chemotherapy, and sometimes during chemotherapy. Radiotherapy may also be administered for palliative reasons to relieve symptoms of cancer, for example, to lessen pain. Among the types of tumors that can be treated using radiotherapy are localized tumors that cannot be excised completely and metastases and tumors whose complete excision would cause unacceptable functional or cosmetic defects or be associated with unacceptable surgical risks.

It will be appreciated that both the particular radiation dose to be utilized in treating lung cancer and the method of administration will depend on a variety of factors. Thus, the dosages of radiation that can be used according to the methods of the present invention are determined by the particular requirements of each situation. The dosage will depend on such factors as the size of the tumor, the location of the tumor, the age and sex of the patient, the frequency of the dosage, the presence of other tumors, possible metastases and the like. Those skilled in the art of radiotherapy can readily ascertain the dosage and the method of administration for any particular tumor by reference to Hall, E. J., Radiobiology for the Radiobiologist, 5th edition, Lippincott Williams & Wilkins Publishers, Philadelphia, Pa., 2000; Gunderson, L. L. and Tepper J. E., eds., Clinical Radiation Oncology, Churchill Livingstone, London, England, 2000; and Grosch, D. S., Biological Effects of Radiation, 2nd edition, Academic Press, San Francisco, Calif., 1980, each of which is incorporated herein by reference.

### M. IMMUNOASSAYS TO MEASURE MARKERS

The methods described above quantify levels of the following combination of markers selected from the group consisting of midkine (MK), tissue factor pathway inhibitor (TFPI), neuron-specific enolase (NSE) and carbohydrate antigen 19-9 (CA19-9). The biomarkers, i.e., MK, TFPI, NSE and CA19-9, may be analyzed for the methods described above using an immunoassay. The presence or amount of marker can be determined using antibodies that specifically bind to each marker (namely, MK, TFPI, NSE and CA19-9 as well as any additional biomarkers if such additional biomarkers are used). Examples of antibodies that can be used include a polyclonal antibody, a monoclonal antibody, a human antibody, an immunoglobulin molecule, a disulfide linked Fv, a monoclonal antibody, an affinity matured, a scFv, a chimeric antibody, a single domain antibody, a CDR-grafted antibody, a diabody, a humanized antibody, a multispecific antibody, a Fab, a dual specific antibody, a DVD, a Fab′, a bispecific antibody, a F(ab′)2, a Fv and combinations thereof. For example, the immunological method may include (a) measuring the levels of MK by: (i) contacting the test sample with at least one capture antibody, wherein the capture antibody binds to an epitope on MK or a fragment of MK to form a capture antibody-MK antigen complex; (ii) contacting the capture antibody-MK antigen complex with at least one detection antibody comprising a detectable label, wherein the detection antibody binds to an epitope on MK that is not bound by the capture antibody and forms a capture antibody-MK-antigen-detection antibody complex; and (iii) determining the MK levels in the test sample based on the signal generated by the detectable label in the capture antibody-MK-antigen-detection antibody complex formed in (a)(ii); (b) measuring the levels of TFPI by: (i) contacting the test sample with at least one capture antibody, wherein the capture antibody binds to an epitope on TFPI or a fragment of TFPI to form a capture antibody-TFPI antigen complex; (ii) contacting the capture antibody-TFPI antigen complex with at least one detection antibody comprising a detectable label, wherein the detection antibody binds to an epitope on TFPI that is not bound by the capture antibody and forms a capture antibody-TFPI antigen-detection antibody complex; and (iii) determining the TFPI levels in the test sample based on the signal generated by the detectable label in the capture antibody-TFPI-antigen-detection antibody complex formed in (b)(ii); (c) measuring the levels of NSE by: (i) contacting the test sample with at least one capture antibody, wherein the capture antibody binds to an epitope on NSE or a fragment of NSE to form a capture antibody-NSE antigen complex; (ii) contacting the capture antibody-NSE antigen complex with at least one detection antibody comprising a detectable label, wherein the detection antibody binds to an epitope on NSE that is not bound by the capture antibody and forms a capture antibody-NSE-antigen-detection antibody complex; and (iii) determining the NSE levels in the test sample based on the signal generated by the detectable label in the capture antibody-NSE-antigen-detection antibody complex formed in (c)(ii); and (d) measuring the levels of CA19-9 by: (i) contacting the test sample with at least one capture antibody, wherein the capture antibody binds to an epitope on CA19-9 or a fragment of CA19-9 to form a capture antibody-CA19-9 antigen complex; (ii) contacting the capture antibody-CA19-9 antigen complex with at least one detection antibody comprising a detectable label, wherein the detection antibody binds to an epitope on CA19-9 that is not bound by the capture antibody and forms a capture antibody-NSE-antigen-detection antibody complex; and (iii) determining the CA19-9 levels in the test sample based on the signal generated by the detectable label in the capture antibody-CA19-9-antigen-detection antibody complex formed in (c)(ii)

Any immunoassay may be utilized. The immunoassay may be an enzyme-linked immunoassay (ELISA), radioimmunoassay (RIA), a competitive inhibition assay, such as forward or reverse competitive inhibition assays, a fluorescence polarization assay, or a competitive binding assay, for example. The ELISA may be a sandwich ELISA. Specific immunological binding of the antibody to the marker can be detected via direct labels, such as fluorescent or luminescent tags, metals and radionuclides attached to the antibody or via indirect labels, such as alkaline phosphatase or horseradish peroxidase.

The use of immobilized antibodies or fragments thereof may be incorporated into the immunoassay. The antibodies may be immobilized onto a variety of supports, such as magnetic or chromatographic matrix particles, the surface of an assay plate (such as microtiter wells), pieces of a solid substrate material, and the like. An assay strip can be prepared by coating the antibody or plurality of antibodies in an array on a solid support. This strip can then be dipped into the test biological sample and then processed quickly through washes and detection steps to generate a measurable signal, such as a colored spot.

The sandwich ELISA measures the amount of antigen between two layers of antibodies (i.e. a capture antibody and a detection antibody (which may be labeled with a detectable label)). The marker, i.e., MK, TFPI, NSE and CA19-9, to be measured may contain at least two antigenic sites capable of binding to antibody. Either monoclonal or polyclonal antibodies may be used as the capture and detection antibodies in the sandwich ELISA.

Generally, at least two antibodies are employed to separate and quantify the marker, i.e., MK, TFPI, NSE and CA19-9 (as well as any additional biomarkers), in a test or biological sample. More specifically, the at least two antibodies bind to certain epitopes of the marker forming an immune complex which is referred to as a “sandwich”. One or more antibodies can be used to capture the marker in the test sample (these antibodies are frequently referred to as a “capture” antibody or “capture” antibodies) and one or more antibodies is used to bind a detectable (namely, quantifiable) label to the sandwich (these antibodies are frequently referred to as the “detection” antibody or “detection” antibodies). In a sandwich assay, both antibodies binding to their epitope may not be diminished by the binding of any other antibody in the assay to its respective epitope. In other words, antibodies may be selected so that the one or more first antibodies brought into contact with a test sample suspected of containing the marker do not bind to all or part of an epitope recognized by the second or subsequent antibodies, thereby interfering with the ability of the one or more second detection antibodies to bind to the marker.

In a preferred embodiment, a test or biological sample suspected of containing the marker, i.e., MK, TFPI, NSE and CA19-9, can be contacted with at least one first capture antibody (or antibodies) and at least one second detection antibodies either simultaneously or sequentially. In the sandwich assay format, a test sample suspected of containing the marker is first brought into contact with the at least one first capture antibody that specifically binds to a particular epitope under conditions which allow the formation of a first antibody-marker complex. If more than one capture antibody is used, a first multiple capture antibody-marker complex is formed. In a sandwich assay, the antibodies, preferably, the at least one capture antibody, are used in molar excess amounts of the maximum amount of marker expected in the test sample.

Optionally, prior to contacting the test sample with the at least one first capture antibody, the at least one first capture antibody can be bound to a solid support which facilitates the separation the first antibody-marker complex from the test sample. Any solid support known in the art can be used, including but not limited to, solid supports made out of polymeric materials in the forms of wells, tubes or beads. The antibody (or antibodies) can be bound to the solid support by adsorption, by covalent bonding using a chemical coupling agent or by other means known in the art, provided that such binding does not interfere with the ability of the antibody to bind the marker. Moreover, if necessary, the solid support can be derivatized to allow reactivity with various functional groups on the antibody. Such derivatization requires the use of certain coupling agents such as, but not limited to, maleic anhydride, N-hydroxysuccinimide and 1-ethyl-3-(3-dimethylaminopropyl)carbodiimide.

After the test sample suspected of containing the marker is brought into contact with the at least one first capture antibody, the test sample is incubated in order to allow for the formation of a first capture antibody (or multiple antibody)-marker complex. The incubation can be carried out at a pH of from about 4.5 to about 10.0, at a temperature of from about 2° C. to about 45° C., and for a period from at least about one (1) minute to about eighteen (18) hours, from about 2-6 minutes, or from about 3-4 minutes.

After formation of the first/multiple capture antibody-marker complex, the complex is then contacted with at least one second detection antibody (under conditions which allow for the formation of a first/multiple antibody-marker second antibody complex). If the first antibody-marker complex is contacted with more than one detection antibody, then a first/multiple capture antibody-marker-multiple antibody detection complex is formed. As with first antibody, when the at least second (and subsequent) antibody is brought into contact with the first antibody-marker complex, a period of incubation under conditions similar to those described above is required for the formation of the first/multiple antibody-marker-second/multiple antibody complex. Preferably, at least one second antibody contains a detectable label. The detectable label can be bound to the at least one second antibody prior to, simultaneously with or after the formation of the first/multiple antibody-marker-second/multiple antibody complex. Any detectable label known in the art can be used.

### N. KITS FOR PERFORMING THE METHODS

Provided herein is a kit, which may be used for performing the methods described above. The kit may provide (1) reagents capable of specifically binding to each of the markers MK, TFPI, NSE and CA19-9, to quantify the levels of the markers, MK, TFPI, NSE and CA19-9, in a biological sample isolated from a subject (2) a reference standard indicating reference levels of each of the markers MK, TFPI, NSE and CA19-9, wherein at least one reagent comprises at least one antibody capable of specifically binding the appropriate marker; and (3) a reference standard indicating a reference level of pack years. The kit may comprise a reagent that is capable of specifically binding to MK, a reagent that is capable of specifically binding to TFPI, a reagent that is capable of specifically binding to NSE and a reagent that is capable of specifically binding to CA19-9 to quantify the concentration of each biomarker in the biological sample and a reference standard indicating the a reference level of each of the biomarker in the biological sample (i.e., MK, TFPI, NSE and CA19-9). The kit may further comprise at least one reagent capable of specifically binding (i.e., an antibody) at least one additional biomarker of nectin-4, cytokeratin 19-fragment 21-1 (CYFRA 21-1), carcinoembryonic antigen (CEA), progastrin releasing peptide (proGRP), carbohydrate antigen 125 (CA125), tissue polypeptide specific antigen (TPS), cancer antigen 15-3 (CA15-3), squamous cell carcinoma antigen (CCA), Helicobacter pylori and/or parainfluenza nectin-4, cytokeratin 19-fragment 21-1 (CYFRA 21-1), carcinoembryonic antigen (CEA), progastrin releasing peptide (proGRP), carbohydrate antigen 125 (CA125), tissue polypeptide specific antigen (TPS), cancer antigen 15-3 (CA15-3), squamous cell carcinoma antigen (CCA), Helicobacter pylori, parainfluenza and a reference standard indicating a reference level of the at least one additional biomarker of lung cancer, if present.

The kit may comprise the antibodies and a means for administering the antibodies. The kit can further comprise instructions for using the kit and conducting the analysis, monitoring, or treatment.

The kit may also comprise one or more containers, such as vials or bottles, with each container containing a separate reagent. The kit may further comprise written instructions, which may describe how to perform or interpret an analysis, monitoring, treatment, or method described herein.

For example, the kit can comprise instructions for assaying the test sample for MK, TFPI, NSE and CA19-9 by immunoassay, e.g., chemiluminescent microparticle immunoassay. The instructions can be in paper form or computer-readable form, such as a disk, CD, DVD, or the like. The antibody can be a MK, TFPI, NSE and CA19-9 capture antibody and/or MK, TFPI, NSE and CA19-9 detection antibody (meaning an antibody labeled with a detectable label). For example, the kit can contain at least one capture antibody that specifically binds MK, at least one capture antibody that specifically binds TFPI, at least one capture antibody that specifically binds NSE and at least one capture antibody that specifically binds CA19-9. The kit can also contain a conjugate antibody (such as an antibody labeled with a detectable label) for each capture antibody (namely, a conjugate antibody for each of the capture antibodies that specifically bind to MK, TFPI, NSE and CA19-9, respectively). Alternatively or additionally, the kit can comprise a calibrator or control, e.g., purified, and optionally lyophilized, (e.g., MK, TFPI, NSE and/or CA19-9), and/or at least one container (e.g., tube, microtiter plates or strips, which can be already coated with an anti-MK, TFPI, NSE and/or CA19-9 monoclonal antibody) for conducting the assay, and/or a buffer, such as an assay buffer or a wash buffer, either one of which can be provided as a concentrated solution, a substrate solution for the detectable label (e.g., an enzymatic label), or a stop solution. Preferably, the kit comprises all components, i.e., reagents, standards, buffers, diluents, etc., which are necessary to perform the assay. The instructions also can include instructions for generating a standard curve or a reference standard for purposes of quantifying MK, TFPI, NSE and CA19-9.

As alluded to above, any antibodies, which are provided in the kit, such as recombinant antibodies specific for MK, TFPI, NSE and CA19-9, can incorporate a detectable label, such as a fluorophore, radioactive moiety, enzyme, biotin/avidin label, chromophore, chemiluminescent label, or the like, or the kit can include reagents for labeling the antibodies or reagents for detecting the antibodies (e.g., detection antibodies) and/or for labeling the analytes or reagents for detecting the analyte. The antibodies, calibrators and/or controls can be provided in separate containers or pre-dispensed into an appropriate assay format, for example, into microtiter plates.

Optionally, the kit includes quality control components (for example, sensitivity panels, calibrators, and positive controls). Preparation of quality control reagents is well-known in the art and is described on insert sheets for a variety of immunodiagnostic products. Sensitivity panel members optionally are used to establish assay performance characteristics, and further optionally are useful indicators of the integrity of the immunoassay kit reagents, and the standardization of assays.

The kit can also optionally include other reagents required to conduct a diagnostic assay or facilitate quality control evaluations, such as buffers, salts, enzymes, enzyme co-factors, substrates, detection reagents, and the like. Other components, such as buffers and solutions for the isolation and/or treatment of a test sample (e.g., pretreatment reagents), also can be included in the kit. The kit can additionally include one or more other controls. One or more of the components of the kit can be lyophilized, in which case the kit can further comprise reagents suitable for the reconstitution of the lyophilized components.

The various components of the kit optionally are provided in suitable containers as necessary, e.g., a microtiter plate. The kit can further include containers for holding or storing a sample (e.g., a container or cartridge for a blood sample). Where appropriate, the kit optionally also can contain reaction vessels, mixing vessels, and other components that facilitate the preparation of reagents or the test sample. The kit can also include one or more instrument for assisting with obtaining a test sample, such as a syringe, pipette, forceps, measured spoon, or the like.

If the detectable label is at least one acridinium compound, the kit can comprise at least one acridinium-9-carboxamide, at least one acridinium-9-carboxylate aryl ester, or any combination thereof. If the detectable label is at least one acridinium compound, the kit also can comprise a source of hydrogen peroxide, such as a buffer, solution, and/or at least one basic solution.

If desired, the kit can contain a solid phase, such as a magnetic particle, bead, test tube, microtiter plate, cuvette, membrane, scaffolding molecule, film, filter paper, a quartz crystal, disc or chip. The kit may also include a detectable label that can be or is conjugated to an antibody, such as an antibody functioning as a detection antibody. The detectable label can for example be a direct label, which may be an enzyme, oligonucleotide, nanoparticle, chemiluminophore, fluorophore, fluorescence quencher, chemiluminescence quencher, or biotin. Kits may optionally include any additional reagents needed for detecting the label.

If desired, the kit can further comprise one or more components, alone or in further combination with instructions, for assaying the test sample for another analyte, which can be a biomarker, such as a biomarker of cancer. Examples of analytes include, but are not limited to MK, TFPI, NSE and CA19-9, and fragments of MK, TFPI, NSE and CA19-9 as well other analytes and biomarkers discussed herein, or otherwise known in the art. In some embodiments one or more components for assaying a test sample for MK, TFPI, NSE and CA19-9 enable the determination of the presence, amount or concentration of MK, TFPI, NSE and CA19-9. A sample, such as a serum sample, can also be assayed for MK, TFPI, NSE and CA19-9 using TOF-MS and an internal standard.

The kit (or components thereof), as well as the method of determining the concentration of MK, TFPI, NSE and CA19-9 in a test sample by an immunoassay as described herein, can be adapted for use in a variety of automated and semi-automated systems (including those wherein the solid phase comprises a microparticle), as described, e.g., in U.S. Pat. Nos. 5,089,424 and 5,006,309, and as commercially marketed, e.g., by Abbott Laboratories (Abbott Park, Ill.) as ARCHITECT®.

Some of the differences between an automated or semi-automated system as compared to a non-automated system (e.g., ELISA) include the substrate to which the first specific binding partner (e.g., analyte antibody or capture antibody) is attached (which can impact sandwich formation and analyte reactivity), and the length and timing of the capture, detection and/or any optional wash steps. Whereas a non-automated format such as an ELISA may require a relatively longer incubation time with sample and capture reagent (e.g., about 2 hours), an automated or semi-automated format (e.g., ARCHITECT® and any successor platform, Abbott Laboratories) may have a relatively shorter incubation time (e.g., approximately 18 minutes for ARCHITECT®). Similarly, whereas a non-automated format such as an ELISA may incubate a detection antibody such as the conjugate reagent for a relatively longer incubation time (e.g., about 2 hours), an automated or semi-automated format (e.g., ARCHITECT® and any successor platform) may have a relatively shorter incubation time (e.g., approximately 4 minutes for the ARCHITECT® and any successor platform).

Other platforms available from Abbott Laboratories include, but are not limited to, AxSYM®, IMx® (see, e.g., U.S. Pat. No. 5,294,404, which is hereby incorporated by reference in its entirety), PRISM®, EIA (bead), and Quantum™ II, as well as other platforms. Additionally, the assays, kits and kit components can be employed in other formats, for example, on electrochemical or other hand-held or point-of-care assay systems. The present disclosure is, for example, applicable to the commercial Abbott Point of Care (i-STAT®, Abbott Laboratories) electrochemical immunoassay system that performs sandwich immunoassays. Immunosensors and their methods of manufacture and operation in single-use test devices are described, for example in, U.S. Pat. No. 5,063,081, U.S. Pat. App. Pub. No. 2003/0170881, U.S. Pat. App. Pub. No. 2004/0018577, U.S. Pat. App. Pub. No. 2005/0054078, and U.S. Pat. App. Pub. No. 2006/0160164, which are incorporated in their entireties by reference for their teachings regarding same.

In particular, with regard to the adaptation of an assay to the I-STAT® system, the following configuration is preferred. A microfabricated silicon chip is manufactured with a pair of gold amperometric working electrodes and a silver-silver chloride reference electrode. On one of the working electrodes, polystyrene beads (0.2 mm diameter) with immobilized capture antibody are adhered to a polymer coating of patterned polyvinyl alcohol over the electrode. This chip is assembled into an I-STAT® cartridge with a fluidics format suitable for immunoassay. On a portion of the wall of the sample-holding chamber of the cartridge there is a layer comprising the detection antibody labeled with alkaline phosphatase (or other label). Within the fluid pouch of the cartridge is an aqueous reagent that includes p-aminophenol phosphate.

In operation, a sample suspected of containing MK, TFPI, NSE and CA19-9 is added to the holding chamber of the test cartridge and the cartridge is inserted into the I-STAT® reader. After the second antibody (detection antibody) has dissolved into the sample, a pump element within the cartridge forces the sample into a conduit containing the chip. Here it is oscillated to promote formation of the sandwich between the first capture antibody, MK, TFPI, NSE or CA19-9, and the labeled second detection antibody. In the penultimate step of the assay, fluid is forced out of the pouch and into the conduit to wash the sample off the chip and into a waste chamber. In the final step of the assay, the alkaline phosphatase label reacts with p-aminophenol phosphate to cleave the phosphate group and permit the liberated p-aminophenol to be electrochemically oxidized at the working electrode. Based on the measured current, the reader is able to calculate the amount of MK, TFPI, NSE or CA19-9 in the sample by means of an embedded algorithm and factory-determined calibration curve.

The present invention has multiple aspects, illustrated by the following non-limiting examples.

It will be readily apparent to those skilled in the art that other suitable modification and adaptations of the methods of the present disclosure described herein are readily applicable and appreciable, and may be made using suitable equivalents without departing from the scope of the present disclosure or the aspects and embodiments disclosed herein. Having now described the present disclosure in detail, the same will be more clearly understood by reference to the following examples which are merely intended only to illustrate some aspects and embodiments of the disclosure, and should not be viewed as limiting to the scope of the disclosure. The disclosures of all journal references, U.S. patents and publications referred to herein are hereby incorporated by reference in their entireties.

### EXAMPLES

### Example 1

Blood samples from normal subjects, subjects with benign lung disease, subjects with stage I lung cancer, subjects with stage II lung cancer, subjects with stage III lung cancer and subjects with stage IV lung cancer were obtained. The total number of subjects was 260. The number of normal subjects was 52 (n=52), the number of subjects with benign lung disease was 106 (n=106), the number of subjects with stage I lung cancer was 38 (n=38), the number of subjects with stage II lung cancer were 14 (n=14) and the number of subjects with stage III and stage IV lung cancer were 50 (n=50). Table 1 below shows the age, sex, race, diagnosis, draw date and number of pack years smoking for the subjects with benign lung disease. Table 2 below shows the age, sex, race, diagnosis, draw date and number of pack years smoking for the subjects with early lung cancer (namely, stage I and stage II). Table 3 below shows the age, sex, race, diagnosis, draw date and number of pack years smoking for the subjects with late stage lung cancer (namely, stage III and stage IV).

TABLE 1

| | | | | Draw | Pack |
|---|---|---|---|---|---|
| Age | Sex | Race | Diagnosis | Date | Years |
| 84 | M | C | BENIGN LUNG DISEASE | Mar. 28, 2003 | 20 |
| 76 | M | C | COPD | Aug. 28, 2003 | 40 |
| 72 | F | C | COPD, EMPHYSEMA | Oct. 20, 2003 | 15 |
| 81 | F | C | PNEUMONIA, MPI | Jan. 6, 2004 | 0 |
| 48 | M | C | BRONCHIECTASIS | Jan. 14, 2004 | 30 |
| 45 | F | C | PULMONARY NODULE, BENIGN | Feb. 5, 2004 | 15 |
| 56 | F | C | COPD, HEMOPTYSIS | Feb. 9, 2004 | 25 |
| 59 | M | B | SARCOIDOSIS | Feb. 11, 2004 | 30 |
| 86 | F | C | ATELECTASIS + COPD | Feb. 27, 2004 | 0 |
| 77 | M | C | COPD | Dec. 27, 2002 | |
| 76 | F | C | COPD | Feb. 11, 2003 | 25 |
| 82 | M | C | COPD | Mar. 10, 2003 | 30 |
| 70 | M | C | COPD | Mar. 12, 2003 | 0 |
| 75 | M | C | COPD | May 28, 2002 | 0 |
| 76 | M | C | ASTHMA | Jul. 7, 2002 | 0 |
| 90 | M | C | COPD | Jul. 21, 2002 | 0 |
| 76 | F | C | COPD | Jul. 17, 2002 | 35 |
| 80 | M | C | ASTHMA | Aug. 4, 2002 | 0 |
| 72 | F | C | COPD | Aug. 6, 2002 | 0 |
| 91 | M | C | ASTHMA | Aug. 15, 2002 | 0 |
| 82 | F | C | ASTHMA | Aug. 29, 2002 | 26 |
| 71 | F | C | ASTHMA | Jun. 6, 2004 | |
| 52 | M | B | ASTHMA | Jul. 8, 2004 | 40 |
| 69 | F | C | COPD | Jan. 6, 2002 | 25 |
| 76 | M | C | COPD | Dec. 20, 2001 | 22 |
| 56 | F | C | ASTHMA | Jan. 7, 2002 | 0 |
| 69 | F | C | COPD | Jan. 6, 2003 | 0 |
| 52 | M | B | ASTHMA | Jul. 15, 2004 | 24 |
| 75 | M | C | ASTHMA | May 23, 2004 | 0 |
| 72 | F | C | ASTHMA | May 24, 2004 | 27 |
| 75 | F | C | ASTHMA | Jun. 14, 2004 | 0 |
| 57 | F | C | ASTHMA | Jun. 28, 2004 | 30 |
| 68 | F | C | ASTHMA | Mar. 17, 2004 | 26 |
| 68 | F | C | ASTHMA | Apr, 15, 2004 | 0 |
| 84 | F | C | ASTHMA | Mar. 4, 2003 | 30 |
| 77 | M | C | COPD | | 40 |
| 71 | F | C | ASTHMA | Mar. 27, 2003 | 0 |
| 70 | F | C | benign lung disease | May 29, 2004 | 0 |
| 73 | F | C | BRONCHITIS | Jun. 10, 2004 | 10 |
| 52 | M | B | ASTHMA | Jun. 11, 2004 | 40 |
| 71 | F | C | BRONCHITIS, ASTHMA | Jul. 5, 2004 | 0 |
| 70 | M | C | benign lung disease | Jun. 30, 2004 | 35 |
| 72 | F | C | benign lung disease | Jun. 30, 2004 | 21 |
| 70 | M | C | benign lung disease | Aug. 11, 2004 | 0 |
| 72 | F | C | ASTHMA | Aug. 27, 2004 | 25 |
| 63 | F | C | ASTHMA | Nov. 23, 2004 | |
| 79 | F | C | COPD | Nov. 24, 2004 | 0 |
| 85 | M | C | COPD | Dec. 3, 2004 | 0 |
| 53 | F | B | COPD, SARCOIDOSIS | Dec. 7, 2004 | 11 |
| 72 | F | C | COPD | Dec. 7, 2004 | 20 |
| 63 | F | C | COPD | Dec. 7, 2004 | 0 |
| 66 | M | C | COPD | Dec. 7, 2004 | 0 |
| 68 | F | C | COPD | Dec. 7, 2004 | 31 |
| 70 | F | C | COPD | Dec. 7, 2004 | 24 |
| 73 | M | C | COPD | Dec. 7, 2004 | 24 |
| 51 | F | C | COPD, EMPHYSEMA | Dec. 8, 2004 | 0 |
| 78 | F | C | COPD, ASTHMA, BRONCHITIS | Dec. 8, 2004 | 24 |
| 51 | F | C | COPD | Dec. 9, 2004 | 37 |
| 58 | F | C | COPD | Dec. 9, 2004 | 0 |
| 60 | M | C | COPD | Dec. 10, 2004 | 39 |
| 74 | F | C | COPD | Dec. 8, 2004 | 24 |
| 55 | F | C | COPD | Dec. 9, 2004 | 54 |
| 56 | M | C | COPD | Dec. 10, 2004 | 22 |
| 54 | F | B | COPD | Dec. 10, 2004 | 22 |
| 57 | M | C | ASTHMA | Dec. 10, 2004 | 29 |
| 94 | F | C | COPD, ASTHMA | Dec. 9, 2004 | 10 |
| 70 | M | C | BENIGN LUNG MASS | Dec. 20, 2004 | |
| 85 | M | C | acute inflammation, COPD | Mar. 20, 2005 | 27 |
| 70 | F | C | chronic inflammation, COPD | Mar. 15, 2005 | 37 |
| 73 | M | C | acute inflammation, COPD | Apr. 4, 2005 | 22 |
| 50 | M | C | chronic inflammation, COPD | Apr. 6, 2005 | 29 |
| 66 | F | C | acute inflammation, COPD | Apr. 17, 2005 | 40+ |
| 58 | F | C | chronic inflammation, COPD | Apr. 19, 2005 | 0 |
| 60 | M | C | dysplasia, COPD | Apr. 20, 2005 | 30+ |
| 70 | F | C | chronic inflammation, COPD | May 2, 2005 | 35+ |
| 70 | F | C | chronic inflammation, emphysema, | May 10, 2005 | 40+ |
| | | | asthma | | |
| 58 | F | C | emphysema, COPD | May 16, 2005 | 35 |
| 53 | M | C | acute inflammation, asthma | May 18, 2005 | 20 |
| 58 | M | C | benign neoplasia, COPD | May 21, 2005 | 40+ |
| 65 | M | C | acute inflammation, COPD | May 26, 2005 | 0 |
| 65 | F | C | COPD | May 30, 2005 | 40+ |
| 62 | F | C | asthma, COPD | Jun. 20, 2005 | 25 |
| 53 | F | C | acute inflammation, asthma | Jun. 22, 2005 | 20+ |
| 59 | F | B | asthma, COPD | Jun. 25, 2005 | 0 |
| 71 | M | C | acute inflammation, COPD | Jun. 30, 2005 | 35+ |
| 78 | F | C | acute inflammation, COPD | Jul. 5, 2005 | 0 |
| 87 | M | C | acute inflammation, COPD | Jul. 9, 2005 | 0 |
| 69 | F | C | COPD | Jul. 11, 2005 | 20 |
| 79 | F | C | COPD | Jul. 11, 2005 | 30+ |
| 54 | M | C | COPD | Jun. 24, 2005 | 10+ |
| 86 | F | C | COPD | Jun. 29, 2005 | 0 |
| 57 | F | C | COPD | Jul. 3, 2005 | 40+ |
| 56 | F | C | COPD | Jul. 6, 2005 | 25+ |
| 52 | F | B | COPD | Jul. 10, 2005 | 0 |
| 70 | F | C | chronic inflammation, COPD | Jul. 11, 2005 | 0 |
| 51 | M | C | COPD | Apr. 6, 2005 | |
| 53 | F | C | asthma | Jul. 7, 2005 | |
| 52 | F | C | asthma | Jul. 10, 2005 | 0 |
| 54 | F | C | asthma, COPD | Jun. 24, 2005 | 10+ |

Sex: M = Male; F = Female
Race: C = Caucasian; B = Black; W = White

TABLE 2

| | | | | Draw | Pack |
|---|---|---|---|---|---|
| Age | Sex | Race | Diagnosis | Date | Years |
| 69 | M | W | LUNG CA-EPIDERMOID, STAGE I | May 28, 2003 | |
| 66 | M | W | LUNG CA-SQUAMOUS CELL, | Jun. 1, 2003 | 40 |
| | | | STAGE I | | |
| 70 | M | C | LARGE CELL CA, STAGE I | Feb. 18, 2004 | 50 |
| 65 | F | C | ADENO, STAGE I | Jan. 28, 2004 | 50 |
| 71 | M | C | ADENO, STAGE I | Oct. 16, 2003 | 30 |
| 71 | F | C | SQUAMOUS, STAGE I | Jan. 13, 2004 | 50 |
| 57 | M | | SQUAMOUS STAGE IB | Jan. 14, 2004 | 0 |
| 68 | M | | SQUAMOUS STAGE IB | Jan. 15, 2004 | 45 |
| 57 | M | C | ADENO, STAGE IB | Jan. 15, 2004 | 40 |
| 74 | F | C | ADENO, STAGE IB | Jan. 15, 2004 | 42 |
| 63 | M | C | LARGE CELL, STAGE IB | Jan. 17, 2004 | 45 |
| 63 | M | A | SQUAMOUS, STAGE IB | Jan. 21, 2004 | 40 |
| 66 | F | C | SQUAMOUS, STAGE IB | Jan. 22, 2004 | 0 |
| 74 | M | C | ADENO, STAGE IB | Jan. 22, 2004 | 50 |
| 73 | M | C | SQUAMOUS, STAGE IB | Jan. 23, 2004 | 53 |
| 72 | M | A | ADENO STAGE IB | Jan. 26, 2004 | n |
| 46 | M | C | SQUAMOUS, STAGE IB | Jan. 27, 2004 | 30 |
| 65 | M | C | ADENO, STAGE IB | Jan. 27, 2004 | 35 |
| 55 | F | C | ADENO, STAGE IB | Jan. 22, 2004 | 50 |
| 72 | M | C | ADENO, STAGE IB | Jan. 29, 2004 | 30 |
| 70 | F | C | SQUAMOUS STAGE I | Apr. 4, 2004 | 0 |
| 70 | F | A | SQUAMOUS, STAGE I | Feb. 4, 2004 | 24 |
| 70 | M | C | SQUAMOUS STAGE IB | Apr. 10, 2004 | 0 |
| 63 | M | C | LARGE CELL STAGE IB | Feb. 10, 2004 | 40 |
| 69 | M | C | LARGE CELL STAGE IB | Feb. 13, 2004 | 50 |
| 78 | M | C | SMALL CELL, STAGE I | Feb. 17, 2004 | 45 |
| 56 | M | C | ADENO, STAGE IB | Feb. 17, 2004 | 30 |
| 56 | M | C | ADENO, STAGE IB | Feb. 17, 2004 | 28 |
| 72 | M | C | ADENO, STAGE IB | Feb. 25, 2004 | 30 |
| 72 | M | C | ADENO, STAGE IB | Feb. 27, 2004 | 30 |
| 65 | F | C | LARGE CELL STAGE IB | Mar. 1, 2004 | 30 |
| 68 | M | A | SQUAMOUS STAGE IB | Mar. 1, 2004 | 35 |
| 70 | M | C | ADENO, STAGE IB | Mar. 2, 2004 | 60 |
| 66 | F | C | ADENO T1N0M0, stage I | Jun. 9, 2003 | 51 |
| 74 | M | C | SQUAMOUS STAGE IB | Nov. 5, 2003 | |
| 84 | M | C | ADENO T1N0M0, stage I | Jul. 13, 2004 | 30 |
| 62 | M | C | ADENO T1N0M0, stage I | Jun. 28, 2004 | 45 |
| 70 | M | C | SQUAMOUS T2N0M0, stage Ib | Jun. 9, 2004 | |
| 69 | F | W | LUNG CA STAGE II ADENO | Apr. 16, 2003 | |
| 76 | F | W | LUNG CA STAGE II SMALL CELL | Jun. 11, 2003 | |
| 73 | M | B | LUNG CA STAGE II ADENO | Jul. 7, 2003 | |
| 71 | F | W | LUNG CA STAGE II ADENO | Aug. 3, 2003 | |
| 60 | F | B | LUNG CA STAGE II SQUAMOUS | Aug. 5, 2003 | |
| 69 | M | W | LUNG CA-EPIDERMOID, STAGE I | May 28, 2003 | |
| 66 | M | W | LUNG CA-SQUAMOUS CELL, | Jun. 1, 2003 | 50 |
| | | | STAGE I | | |
| 67 | M | B | SQUAMOUS, STAGE II | Nov. 3, 2003 | 50 |
| 73 | M | B | SQUAMOUS, STAGE II | Nov. 6, 2003 | 44 |
| 54 | M | C | SQUAMOUS, STAGE IIB | Jan. 17, 2004 | 50 |
| 65 | M | C | ADENO, STAGE IIB | Jan. 20, 2004 | 40 |
| 74 | F | C | EPIDERMOID, T3NXM0, stage II | Nov. 28, 2003 | |
| 79 | F | C | ADENO, STAGE II | Sep. 29, 2003 | 45 |
| 75 | M | C | SQUAMOUS T2N0Mx, stage 2 | Aug. 9, 2004 | |

Sex: M = Male; F = Female
Race: C = Caucasian; B = Black; W = White

TABLE 3

| | | | | Pack |
|---|---|---|---|---|
| Age | Sex | Race | Diagnosis | Years |
| 74 | M | C | Late stage lung cancer | |
| 73 | M | C | Late stage lung cancer | |
| 67 | F | B | Late stage lung cancer | |
| 65 | F | B | Late stage lung cancer | |
| 71 | M | C | Late stage lung cancer | |
| 69 | M | C | Late stage lung cancer | |
| 67 | M | C | Late stage lung cancer | |
| 55 | M | C | Late stage lung cancer | |
| 57 | M | C | Late stage lung cancer | |
| 52 | M | C | Late stage lung cancer | |
| 65 | M | C | Late stage lung cancer | |
| 73 | M | C | Late stage lung cancer | |
| 76 | M | C | Late stage lung cancer | |
| 75 | M | C | Late stage lung cancer | |
| 50 | M | C | Late stage lung cancer | |
| 74 | M | C | Late stage lung cancer | |
| 62 | M | C | Late stage lung cancer | |
| 57 | F | C | Late stage lung cancer | |
| 69 | M | C | Late stage lung cancer | |
| 60 | F | C | Late stage lung cancer | |
| 55 | F | C | Late stage lung cancer | |
| 52 | M | C | Late stage lung cancer | |
| 57 | M | C | Late stage lung cancer | |
| 74 | M | C | Late stage lung cancer | 35 |
| 68 | F | H | Late stage lung cancer | 45 |
| 72 | M | C | Late stage lung cancer | 25 |
| 74 | M | C | Late stage lung cancer | 47 |
| 71 | M | C | Late stage lung cancer | |
| 60 | F | C | Late stage lung cancer | 28 |
| 52 | M | C | Late stage lung cancer | 0 |
| 27 | F | | Late stage lung cancer | |
| 84 | M | C | Late stage lung cancer | 50 |
| 66 | M | C | Late stage lung cancer | 0 |
| 55 | M | C | Late stage lung cancer | 0 |
| 57 | M | C | Late stage lung cancer | 0 |
| 62 | F | C | Late stage lung cancer | 40 |
| 62 | M | C | Late stage lung cancer | 36 |
| 79 | M | | Late stage lung cancer | 0 |
| 55 | M | | Late stage lung cancer | 45 |
| 55 | M | | Late stage lung cancer | 28 |
| 72 | M | | Late stage lung cancer | 36 |
| 78 | M | | Late stage lung cancer | 0 |
| 66 | F | | Late stage lung cancer | 35 |
| 76 | M | | Late stage lung cancer | 0 |
| 53 | F | | Late stage lung cancer | 0 |
| 60 | M | | Late stage lung cancer | 37 |
| 67 | F | | Late stage lung cancer | 30 |
| 68 | M | | Late stage lung cancer | 0 |
| 29 | F | | Late stage lung cancer | |
| | | | Late stage lung cancer | |
| | | | Late stage lung cancer | |
| | | | Late stage lung cancer | |
| | | | Late stage lung cancer | |
| | | | Late stage lung cancer | |
| | | | Late stage lung cancer | |
| | | | Late stage lung cancer | |

Sex: M = Male; F = Female
Race: C = Caucasian; B = Black; W = White

The purpose of this study was to evaluate a series of biomarkers in combination with the number of pack years smoking could be used to differentiate subjects suffering from (1) benign lung disease from lung cancer stages I and II; and (2) benign lung disease from lung cancer stage I. The markers tested were: Midkine, NSE, CYRFA21-1, TPS, CEA, CA125, proGRP, Nectin-4, SCC, CA15-3, CA19-9, TFPI, H. pylori and parainfluenza.

The below assay formats used to detect each biomarker is shown in Table 4 below: FIG. 10 shows the ARCHITECT® (Abbott Laboratories, Abbott Park, Ill.) sandwich immunoassay used in this study. Briefly, in this immunoassay, an antibody coated on a microparticle captures the analyte of interest, then a second antibody conjugated to acridinium binds to a second epitope on the analyte, then a separation of the particles from the label and subsequent read is performed. FIG. 11 shows the enzyme-linked immunoassays (ELISA) used in this study. The ELISAs use an enzyme to quantitate the amount of protein biomarker in a serum sample. Specifically, FIG. 11 shows a typical sandwich ELISA in which: (1) Plate is coated with a capture antibody; (2) sample is added, and any antigen (protein biomarker for pancreatic cancer) present binds to capture antibody; (3) detecting antibody is added, and binds to antigen; (4) enzyme-linked secondary antibody is added, and binds to detecting antibody; (5) substrate is added, and is converted by enzyme to a detectable form (http://en.wikipedia.org/wiki/File:ELISA-sandwich.svg). In this study, the ELISA assays were run in accordance of the manufacturer's instructions.

TABLE 4

| Assay Format | Biomarker |
|---|---|
| ELISA | Midkine |
| ELISA | NSE |
| ARCHITECT ® | CYRFA 21-1 |
| ELISA | TPS |
| ARCHITECT ® | CEA |
| ARCHITECT ® | CA125 |
| ELISA | proGRP |
| ELISA | Nectin-4 |
| ARCHITECT ® | SCC |
| ARCHITECT ® | CA15-3 |
| ARCHITECT ® | CA19-9 |
| ELISA | TFPI |
| ELISA | H. pylori |
| ELISA | Parainfluenza |

The MK dot plot shows differentiation of the normal and benign from the early and late cancers. This is also evident in the AUROC analysis where MK had an AUROC of 0.78 when comparing benign versus stage I and II lung cancers. The differences on the dot plots between the non-cancer versus the early and late lung cancer are less pronounced for TFPI, NSE and CA19-9 (See, FIGS. 2-4). However, in the multivariate models the panel of biomarkers MK, TFPI, NSE, CA19-9 plus pack years significantly improves the AUROC for differentiating benign disease from stage I cancer, stage I and II cancers and all cancers combined. The AUROC for the benign versus stage I perform equally well as the benign versus stage I and II for the combined panel (See, Table 5). Even though there are other biomarkers with a better AUROC than CA19-9 and TFPI, their contribution is in the panel with strength in correctly detecting benign disease. The dot plots were made using Microsoft® Office Excel 2003 plus Analyse-it for Excel version 2.12. Univariate and multivariate analysis using JMP version 9.0.0 was conducted on the biomarker data.

TABLE 5
Univariate AUROC: Early Detection of Pancreatic Cancer Study

| | | Benign | | Non- |
|---|---|---|---|---|
| | | vs | Benign | Cancer |
| | | Stage | vs | vs |
| Assay Format | Biomarker | I & II | Cancer | Cancer |
| Biovendor/ELISA | Midkine | 0.78 | 0.79 | 0.80 |
| ELISA | NSE | 0.76 | 0.78 | 0.79 |
| Abbott/ARCHITECT | CYFRA 21-1 | 0.70 | 0.70 | 0.71 |
| ELISA | TPS | 0.69 | 0.69 | 0.70 |
| Abbott/ARCHITECT | CEA | 0.62 | 0.68 | 0.68 |
| Abbott/ARCHITECT | CA125 | 0.62 | 0.67 | 0.70 |
| ELISA | proGRP | 0.61 | 0.65 | 0.67 |
| USCN/ELISA | Nectin 4 | 0.60 | 0.63 | 0.64 |
| Abbott/ARCHITECT | SCC | 0.60 | 0.61 | 0.62 |
| Abbott/ARCHITECT | CA15-3 | 0.56 | 0.59 | 0.62 |
| Abbott/ARCHITECT | CA19-9 | 0.55 | 0.53 | 0.54 |
| R&D Systems/ELISA | TFPI | 0.53 | 0.52 | 0.52 |
| ELISA | H Pylori | 0.80 | 0.78 | 0.80 |
| ELISA | Parainfluenza | 0.61 | 0.61 | 0.61 |

Multivariate AUROC: Early Detection of Pancreatic Cancer Study

| | | Benign | | |
|---|---|---|---|---|
| | | vs | Benign | Benign |
| | | Stage | vs | vs |
| Assay Format | Biomarker | I & II | Stage I | Cancer |
| Patient Clinical Data | Pack Years | 0.92 | 0.92 | 0.93 |
| Biovendor/ELISA | Midkine | | | |
| ELISA | NSE | | | |
| R&D Systems/ELISA | TFPI | | | |
| Abbott/ARCHITECT | CA19-9 | | | |

| Specimen | n |
|---|---|
| Normal | 52 |
| Benign | 106 |
| Stage I | 38 |
| Stage II | 14 |
| Stage III & IV | 50 |
| Total | 260 |

JMP software version 9.0.0 Copyright © 2010 SAS Institute Inc.

### Example 2

Blood samples from subjects with benign lung disease and early lung cancer (Stage I and II). The total number of subjects was 106. The number subjects with benign lung disease was 52 (n=52), the number of subjects with early lung cancer was 54 (n=54). The objective of this study was to assess three markers, TFPI, Midkine and Nectin-4 for their ability to discriminate between benign lung disease and early lung cancer. A secondary objective was to combine these three markers with other existing markers and assess their ability to discriminate between benign conditions and early lung cancer. For the secondary objections, not all subjects had results for every marker explored. Many of the analyses performed below account for missing data either via imputation or through validation techniques that create multiple data sets.

Statistical Evaluation

Distributions of the variables between the benign and early cancer group were evaluated. In order to assess new biomarkers, one essentially asks whether the new marker predicts more accurately than other markers. Three statistical measures were employed to evaluate this question: discrimination, calibration and global fit. These are described in more detail below.

Validation Evaluation

Several methods were used in order to validate the statistical models. These included resubstitution, split-sample validation, cross validation evaluations such as leave-one-out and k-fold cross validation and bootstrapping. These are described in more detail below.

Multivariable Statistical Evaluation

Several methods were used to evaluate several markers simultaneously. These included Principal Component Analysis, Hierarchical Clustering Analysis, Linear Discriminant Analysis, and Partition Analysis. These are described in more detail below.

Statistical Evaluation: Multiple Testing

In order to detect a potentially significant differential response in a large set of variables between the outcome groups, the hypothesis test of each individual variable was adjusted to reduce the possibility of spurious findings. The multiple-hypotheses testing problem was addressed through the estimation of the false discovery rate (FDR). FDR methods aim to increase the power in statistical testing. The positive FDR (pFDR) method was chosen. This method computes the “q-values”, which are adaptive adjusted p-values. These adjusted p-values are smaller than raw p-values, and by controlling the false discovery rate, hypothesis tests at a level less than the observed p-value can be rejected.

The output displays the mean and standard deviation for every variable, by outcome group as shown below in Table 6:

TABLE 6
Continuous Variable Tabulations

| | | Standard | | |
|---|---|---|---|---|
| Variable | Group 1 | NumObs | Mean | Deviation |
| TFPI | Benign | 106 | 29.4449 | 8.3109 |
| TFPI | Early | 52 | 30.4221 | 11.6376 |
| LnMK | Benign | 99 | −2.3204 | 1.4454 |
| LnMK | Early | 51 | −1.2131 | 1.0137 |
| MK | Benign | 106 | 0.6575 | 3.3807 |
| MK | Early | 52 | 0.5365 | 0.9913 |
| Nectin-4 | Benign | 106 | 10.7901 | 25.5936 |
| Nectin-4 | Early | 52 | 26.4813 | 56.6097 |
| CA125 | Benign | 97 | 20.4205 | 14.3422 |
| CA125 | Early | 52 | 51.8620 | 126.5080 |
| CA15-3 | Benign | 97 | 20.2408 | 25.3611 |
| CA15-3 | Early | 52 | 35.4358 | 105.9473 |
| CA19-9 | Benign | 97 | 20.7363 | 22.4397 |
| CA19-9 | Early | 52 | 63.9048 | 333.4465 |
| CEA | Benign | 97 | 6.2666 | 35.9189 |
| CEA | Early | 52 | 12.3257 | 39.3435 |
| CYFRA21-1 | Benign | 94 | 2.8255 | 9.1731 |
| CYFRA21-1 | Early | 52 | 6.8181 | 12.8381 |
| H. pylori | Benign | 97 | 33.8115 | 89.5943 |
| H. pylori | Early | 52 | 157.1319 | 324.9024 |
| NSE | Benign | 46 | 2.1881 | 1.3836 |
| NSE | Early | 49 | 6.9019 | 19.3380 |
| Parainfluenza | Benign | 44 | 150.2841 | 161.1888 |
| Parainfluenza | Early | 49 | 205.7696 | 845.5328 |
| SCC | Benign | 97 | 1.9859 | 4.1267 |
| SCC | Early | 52 | 2.6233 | 6.4989 |
| TPS | Benign | 46 | 108.4224 | 315.5056 |
| TPS | Early | 49 | 164.7427 | 225.1984 |
| proGRP | Benign | 46 | 10.5925 | 8.0542 |
| proGRP | Early | 49 | 40.9698 | 168.8596 |
| pack years | Benign | 80 | 15.9000 | 14.9815 |
| pack years | Early | 42 | 37.4048 | 14.9129 |
| age | Benign | 98 | 67.4388 | 11.9285 |
| age | Early | 52 | 67.0000 | 7.1647 |

The outcome further displays the raw p-value and the p-value after adjustment (See, FIG. 5). Prior to the adjustment method, MK (log transformed), Nectin-4, CA 125, CYFRA 21-1, H. pylori, and pack years of smoking (pack years) are significantly different between the benign and early cancer groups. After adjustment, many values are significantly different between the benign lung disease (benign) and early cancer groups, or borderline statistical significance.

Statistical Evaluation: Discrimination Analysis

Discrimination for the purposes of this study is defined as how well the model separates individuals who develop the outcome from those who do not.

As used in this study, the term C-statistic (AUC) refers to the probability that a randomly selected person with the event will have a higher predicted risk than a randomly selected person without the event. A c-statistic of 1 indicates perfect discrimination and 0.5 indicates no predictive ability.

Table 7 below provides the c-statistic (AUC) for the markers comparing benign versus early cancer.

TABLE 7

| Marker | AUC |
|---|---|
| TFPI | 0.467 |
| MK | 0.208 |
| LNMK¹ | 0.793 |
| Nectin-4 | 0.578 |
| CA 125 | 0.617 |
| CA 15-3 | 0.556 |
| CA 19-9 | 0.470 |
| CEA | 0.624 |
| H. Pylori | 0.793 |
| NSE | 0.765 |
| Parainfluenza | 0.391 |
| SCC | 0.613 |
| TPS | 0.689 |
| proGRP | 0.615 |
| Pack years | 0.858 |
| Age | 0.523 |

¹LNMK is the natural log of MK

Statistical Evaluation: Calibration Analysis

For purposes of this study, calibration is defined as how close the predicted risks are to actual observed risks. The Hosmer-Lemeshow (HL) test was used. The Hosmer-Lemeshow test is a statistical test for goodness of fit for logistic regression models. It is used frequently in risk prediction models. The test assesses whether or not the observed event rates match expected event rates in subgroups of the model population. The Hosmer-Lemeshow test specifically identifies subgroups as the deciles of fitted risk values. Models for which expected and observed event rates in subgroups are similar are called well calibrated. (http://en.wikipedia.org/wiki/Hosmer%E2%80%93Lemeshow_test)

For the HL test, higher p values indicate better calibration.

Several markers were well calibrated, meaning that the model form can predict the observed pattern (specifically, TFPI, Nectin-4, CA 125, CA 15-3, CA 19-9, proGRP). CA 15-3 is the best calibrated by this measure. However, only CA 125 and NSE were found to be statistically associated with early cancer. LNMK and pack years are strongly associated with cancer, but this method does not appear to be able to predict the observed pattern. When looking at the distribution of LNMK by outcome, the distribution appears bi-modal, rather than continuous, due to the “outliers”, even though log transformed. As a result, the HL test would be a weak test for this type of apparent distribution.

Statistical Evaluation: Global Fit

For this study, global measures of model fit is defined as assessing how likely that the model chosen would give rise to the data observed.

Aikaike Information Criteria (AIC) is used to determine how likely the model chosen would give the observed data. This measure takes into account the number of variables in the predictive model. Model with fewer variables would be preferred if the alternative model provides equally good predictions. For the AIC, lower values are better and a general rule of thumb is that a change in AIC of 2 units is statistically significant.

As shown in Table 8 below, both NSE and pack years provide the best fit in explaining the variation seen in the data by this method.

TABLE 8

| | | 95% CI | | | | | |
|---|---|---|---|---|---|---|---|
| Model | OR | Lower | Upper | AUC | HL | AIC | |
| TFPI | 1.01 | 0.98 | 1.05 | 0.467 | 0.2894 | 203.834 | |
| MK | 0.98 | 0.87 | 1.12 | 0.208 | 0.0001 | 204.134 | |
| LNMK | 1.95 | 1.41 | 2.68 | 0.793 | 0.0098 | 173.940 | * |
| Nectin-4 | 1.01 | 1.00 | 1.02 | 0.578 | 0.3186 | 198.838 | |
| CA 125 | 1.03 | 1.01 | 1.04 | 0.617 | 0.3527 | 183.920 | * |
| CA 15-3 | 1.00 | 1.00 | 1.01 | 0.556 | 0.7603 | 190.915 | |
| CA 19-9 | 1.00 | 1.00 | 1.01 | 0.470 | 0.2405 | 195.072 | |
| CEA | 1.00 | 1.00 | 1.01 | 0.624 | 0.0374 | 195.897 | |
| H. Pylori | 1.01 | 1.00 | 1.01 | 0.793 | <0.0001 | 181.913 | |
| NSE | 1.71 | 1.25 | 2.34 | 0.765 | 0.2460 | 115.088 | * |
| Para- | 1.00 | 1.00 | 1.00 | 0.391 | 0.1174 | 132.461 | |
| influenza | | | | | | | |
| SCC | 1.02 | 0.96 | 1.09 | 0.613 | 0.0959 | 196.238 | |
| TPS | 1.00 | 1.00 | 1.00 | 0.689 | 0.0067 | 134.489 | |
| proGRP | 1.04 | 1.00 | 1.09 | 0.615 | 0.2799 | 129.553 | |
| Pack years | 1.10 | 1.06 | 1.14 | 0.858 | 0.0033 | 115.197 | * |
| Age | 1.00 | 0.97 | 1.03 | 0.523 | 0.0026 | 197.548 | |

Validation Evaluation

For the validation evaluation, the following definitions were used:

Resubstitution: measures of model performance are computed from the same data that is used for model fitting. This is also known as “double-dipping” since the data is used twice, one for fitting and once for predicting. Measures of model performance tend to reflect optimistic predictions because they indicate better accuracy than the actual model would allow in practice.

Split-sample: data is split into a training dataset and test dataset. The training dataset is used to fit the model and the performance of this model is evaluated using the test dataset. Model performance is often less biased.

Leave-one-out: a sample reuse method that increases the efficiency by repeated use of observed data points, estimating the model performance with smaller variance, but an increase in bias since each observation is used more than once. In this method, one observation of the dataset is left out and a model is built on the remaining observations. The model is then used to predict the left-out sample. This process is repeated for each observation, resulting in a new dataset with a predicated probability for each observation and which is used to construct the cross-validated discrimination measure.

K-fold cross validation: another sample reuse sample method that breaks the data into k randomly chosen segments. The analysis is repeated k times. For each of the analyses, one of the k segments is used as the test dataset and the other k−1 segments are used as the training datasets. One prediction for each observation forms the basis of the k-fold cross validated discrimination measure.

Bootstrapping: another sample resuse method that can result in lower variances as a result of forming B bootstrap samples, where B is typically greater than 100. B bootstrap samples are formed and used as the training sample. The original data as well as the bootstrap sample are then used as test samples. The difference in the discrimination measure estimated from the original sample and from the bootstrap method is a measure of optimism. This optimism value can be subtracted from the resubstitution measure of discrimination to produce an optimism-corrected measure of discrimination.

Table 9 shows each of the methods used in the validation evaluation.

TABLE 9

| | | Training | | | | Test Validation | | |
|---|---|---|---|---|---|---|---|---|
| | | | 95% CI | | | | 95% CI | |
| Method | Model | OR | Lower | Upper | AUC | AUC | Lower | Upper |
| Resubstitution | All Markers | | | | 0.99 | | | |
| | Pack years + | | | | 0.92 | | | |
| | LNMK + TFPI | | | | | | | |
| Split-Sample | Pack years | 1.99 | 1.14 | 3.46 | 0.89 | 0.89 | 0.77 | 1.01 |
| | LNMK | 1.10 | 1.04 | 1.15 | | | | |
| Leave One | Pack years | 1.10 | 1.06 | 1.17 | 0.92 | 0.86 | 0.79 | 0.94 |
| Out | LNMK | 2.87 | 1.57 | 5.25 | | | | |
| | TFPI | 0.90 | 0.81 | 0.99 | | | | |
| K-Fold | Pack years + | | | | 0.91 | | | |
| | LNMK | | | | | | | |
| | Pack years + | | | | 0.93 | | | |
| | LNMK + CA | | | | | | | |
| | 19-9 + TFPI | | | | | | | |
| | Pack years + | | | | 0.94 | | | |
| | LNMK | | | | | | | |
| | Pack years + | | | | 0.97 | | | |
| | LNMK + NSE + | | | | | | | |
| | TFPI | | | | | | | |
| | Pack years + | | | | 0.91 | 0.84 | 0.75 | 0.92 |
| | LNMK | | | | | | | |
| Bootstrapping | All Markers | | | | 0.99 | 0.92 | | |
| | Pack years + | | | | 0.90 | 0.89 | | |
| | LNMK | | | | | | | |
| | Pack years + | | | | 0.90 | 0.88 | | |
| | LNMK + CA | | | | | | | |
| | 19-9 + TFPI | | | | | | | |
| | Pack years + | | | | 0.97 | 0.96 | | |
| | LNMK + NSE + | | | | | | | |
| | TFPI | | | | | | | |

For the models above, all of the markers were considered unless otherwise noted in the text. The best model was selected using forward stepwise selection methodology. A summary of the evaluation is provided below:

Resubstitution: All markers create a fully estimated model with an AUC=0.99. However, using forward stepwise methods, the model that met the statistical inclusion criteria included Pack years, LNMK and TFPI. As noted before, resubstitution often results in an overly fitted, overly optimistic model. With this model, the AUC=0.92. The bias seen in resubstitution can often be reduced by using the split sample procedure.

Split-Sample: The predictive model is built on the training dataset and the estimates for the model discrimination are obtained using the test dataset. In order to estimate the model discrimination, the predicted probabilities obtained from the fitted model are used as the marker values, resulting in a corrected AUC=0.89. The lower AUC found with this method illustrates the overestimation of the resubstitution method. The best model built using this model included Pack years and LNMK.

Leave One Out: Similar results are seen with this validation method. An AUC of 0.92 is corrected to 0.86 using the cross-validated sample, producing a model with Pack years, LNMK and TFPI.

K-Fold: Using a 5-fold cross validation method one of the 5 segments is left out as the test set and the remaining 4 sets are used for training, producing 5 models. Three models built an optimal model including Pack years and LNMK, another of Pack years, LNMK, CA 19-9, and TFPI and another of Pack years, LNMK, NSE and TFPI. AUCs ranged from 0.91 to 0.97. The final cross-validated discrimination measure results in an AUC of 0.84.

Bootstrapping: 100 bootstraps were performed. The measure of optimism is subtracted form the resubstitution AUC to produce an optimism-corrected AUC. With this method a validated measure of discrimination was calculated as AUC=0.92 when all markers were allowed to be selected into the model. Three other models (as found through the K-fold method) were bootstrapped, with validated measures of discrimination ranging from 0.88 to 0.96.

As shown in FIG. 6, an exploratory principal component analysis was performed. From FIG. 6 and loading scores, up to 8 groupings of markers were made:
CA 15-3, CA 125
CYFRA 21-1, TPS
Nectin-4, CEA
NSE, proGRP
CA 19-9
TFPI
Pack years
LNMK
SCC, Parainfluenza, H. pylori and age did not group into any pattern

From the models constructed in the validation procedures, the markers Pack years, LNMK, TFPI, NSE and CA 19-9 at times were selected. From the principal components analysis, all of these markers are independent.

Multivariable Statistical Evaluation: Hierarchical Clustering Analysis

A hierarchical clustering analysis of the markers was performed as shown in FIG. 7 and the results shown below:

| Number of | | | |
|---|---|---|---|
| Clusters | Distance | Leader | Joiner |
| 15 | 1.13196899 | NSE | proGRP |
| 14 | 2.26985930 | CA15_3 | CA125 |
| 13 | 2.42561343 | CYFRA_21_1 | TPS_{—} |
| 12 | 4.69811819 | Nectin_4 | CEA |
| 11 | 6.54108915 | CA19_9 | CA15_3 |
| 10 | 7.16851050 | TFPI | LNMK |
| 9 | 7.75160340 | CA19_9 | SCC |
| 8 | 7.89739075 | H_Pylori | pack_yrs |
| 7 | 8.22570969 | Nectin_4 | CYFRA_21_1 |
| 6 | 8.44360075 | Parainfluenza | age |
| 5 | 8.69730064 | TFPI | H_Pylori |
| 4 | 10.13843912 | Nectin_4 | Parainfluenza |
| 3 | 11.29480249 | TFPI | CA19_9 |
| 2 | 11.65677375 | TFPI | Nectin_4 |
| 1 | 12.50963107 | TFPI | NSE |

As discussed above in connection with the principal components analysis, 8 groupings of markers were found, now labeled with the cluster determination from the cluster analysis:
CA 15-3, CA 125 (cluster 14)
CYFRA 21-1, TPS (cluster 13)
Nectin-4, CEA (cluster 12)
NSE, proGRP (cluster 15)
CA 19-9 (cluster 11)
TFPI (cluster 10)
Pack years (cluster 8)
LNMK (cluster 10)

In this analysis, the clusters most related were also determined by the principal component analysis.

Multivariable Statistical Evaluation: Discriminant Analysis

A linear discriminant analyses were performed. Using a stepwise approach, a linear combination of all markers misclassified 6.6% observations (n=5); however, the sample size was greatly reduced to n=76 with observations that have all marker values.

Counts: Actual Rows by Predicted Columns

| | Benign | Early |
|---|---|---|
| Benign | 33 | 3 |
| Early | 2 | 38 |

Using only the variables that have been highlighted in the validation models (except NSE, due to the fact that is has a large amount of missing data), n=20 (17.5%) of the data was misclassified from a total dataset of n=114.

Counts: Actual Rows by Predicted Columns

| | Benign | Early |
|---|---|---|
| Benign | 59 | 14 |
| Early | 6 | 35 |

Also, the plot shown in FIG. 8 indicated that Pack years and LN MK are markers of early disease, while TFPI and CA 19-9 lean towards benign. This was not surprising given their distributions. For both of these markers, the distributions of those with benign disease and those with early disease were overlapping, and both were right skewed (highest values among those with early disease). However, for both markers the median of the benign group was higher than that of the early group. As a result, the markers would have a statistical tendency toward the benign.

Multivariable Statistical Evaluation: Partition Analysis

Partition analysis was performed to identify markers that might be associated with early cancer. This analysis produces a single tree, growing by splitting at optimum splits. Optimum splits are found by considering every possible X variable. The variable with the largest LogWorth result is identified as the optimum split. Trees with several splits can “overfit” the data. The model predicts the fitted data very well; however predicts future subjects poorly. As a result, a process of validation should be used. In this step, a part of the data (‘training’ set) is used to estimate the model parameters, and the other part (‘validation’ set) is used to assess the predictive ability of the model. One typical validation method includes the holdback method, in which 80% of the data would be allotted to the training set and 20% of the data would be allotted to the validation set. Another typical validation method is the kfold method. In this analysis, a 5-fold cross-validation method was chosen, as it more efficient for small sample sizes. This method divides the data into k subsets, and each of the k subsets are used to validate the model fit on the other (k−1) data, fitting a total of k models. The model giving the best validation statistic is chosen as the final model.

The 5-fold cross validation produced a model that split 14 times, and pruned back to only 5 splits, for a cross-validates R² value of 0.52. The first variable that was used to split the data was Pack years. As shown in FIG. 9, persons with more pack years are more likely to have early cancer (hash marks) and those with lower values are more likely to be benign (gray with no hash marks). Among several performances of this partition analysis, persons with lower amounts of pack years have always used the marker LNMK to split next, with those with small values more likely to be benign and those with larger values more likely to be early stage.

Although the partition shows that among pack users with more years, the next split is CYFRA, repetitive analyses of partitions have used H. pylori, NSE or age (this split was not stable).

The results show that the combination of pack years of smoking and MK are useful to discriminate between benign lung disease and early stage lung cancer. Adding additional markers, such as TFPI, NSE or CA 19-9 may also be useful.

What is claimed is:

1. A method for identifying and treating a subject having or at risk of having lung cancer, the method comprising the steps of:
a. obtaining a biological sample from a subject;
b. determining the levels of midkine (MK), tissue factor pathway inhibitor (TFPI), neuron-specific enolase (NSE) and carbohydrate antigen 19-9 (CA19-9) in the biological sample from the subject;
c. determining the number of pack years of smoking of the subject;
d. comparing the levels of MK, TFPI, NSE and CA19-9 in the biological sample to reference levels of MK, TFPI, NSE and CA19-9;
e. comparing the number of pack years of smoking by the subject to a reference level of pack years;
f. identifying the subject as having lung cancer or at risk of having lung cancer if the levels of MK, TFPI, NSE and CA19-9 in the biological sample are greater than the reference levels of MK, TFPI, NSE and CA19-9 and the number of pack years of smoking by the subject is greater than the reference level of pack years; and
g. administering a lung cancer treatment regimen to the subject identified as having lung cancer or a lung monitoring regimen to the subject identified as at risk of having lung cancer.

2. The method of claim 1, wherein the lung cancer is early lung cancer.

3. The method of claim 2, wherein the early lung cancer is Stage I or Stage II non-small cell lung cancer or limited stage small cell lung cancer.

4. The method of claim 1, wherein the reference levels of MK, TFPI, NSE and CA19-9 are the MK, TFPI, NSE and CA19-9 cutoff values determined by a receiver operating curve (ROC) analysis from biological samples of a patient group.

5. The method of claim 1, wherein the reference levels of MK, TFPI, NSE and CA19-9 are the MK, TFPI, NSE and CA19-9 cutoff values determined by a quartile analysis of biological samples of a patient group.

6. The method of claim 1, wherein the MK, TFPI, NSE and CA19-9 reference level is higher than or equal to 0.05 ng/mL, 0.06 ng/mL, 0.07 ng/mL, 0.08 ng/mL, 0.09 ng/mL, 0.10 ng/mL, 0.20 ng/mL, 0.30 ng/mL or 0.40 ng/mL in serum for MK in combination with levels higher than or equal 20 pg/mL, 21 pg/mL, 22 pg/mL, 23 pg/mL, 24 pg/mL, 25 pg/mL, 26 pg/mL, 27 pg/mL, 28 pg/mL, 29 pg/mL, 30 pg/mL, 31 pg/mL, 32 pg/mL, 33 pg/mL, 34 pg/mL, 35 pg/mL, 36 pg/mL, 37 pg/mL, 38 pg/mL, 39 pg/mL or 40 pg/mL in serum for TFPI, levels higher than or equal to 1 ng/mL, 2 ng/mL, 3 ng/mL, 4 ng/mL or 5 ng/mL in serum for NSE and levels higher than or equal to 10 U/mL, 11 U/mL, 12 U/mL, 13 U/mL, 14 U/mL, 15 U/mL, 16 U/mL, 17 U/mL, 18 U/mL, 19 U/mL, 30 U/mL, 31 U/mL, 32 U/mL, 33 U/mL, 34 U/mL, 35 U/mL, 36 U/mL or 37 U/mL in serum for CA19-9.

7. The method of claim 1, further comprising determining the level of at least one additional biomarker of lung cancer in the biological sample selected from the group consisting of: nectin-4, cytokeratin 19-fragment 21-1 (CYFRA 21-1), carcinoembryonic antigen (CEA), progastrin releasing peptide (proGRP), carbohydrate antigen 125 (CA125), tissue polypeptide specific antigen (TPS), cancer antigen 15-3 (CA15-3), squamous cell carcinoma antigen (CCA), Helicobacter pylori, parainfluenza and combinations thereof, and comparing the level of the at least one additional biomarker of lung cancer to a reference level for the at least one biomarker of lung cancer.

8. The method of claim 1, wherein the subject is a human.

9. The method of claim 1, wherein the biological sample of a subject is selected from a tissue sample, bodily fluid, whole blood, plasma, serum, urine, bronchoalveolar lavage fluid, and a cell culture suspension or fraction thereof.

10. The method of claim 1, wherein the biological sample of a subject is blood plasma or blood serum.

11. The method of claim 1, wherein determining the levels of MK, TFPI, NSE and CA19-9 comprises an immunological method with molecules specifically binding to MK, TFPI, NSE or CA19-9.

12. The method of claim 11, wherein the molecules specifically binding to MK, TFPI, NSE and CA19-9 comprises at least one antibody capable of specifically binding MK, TFPI, NSE or CA19-9.

13. The method of claim 7, wherein levels MK, TFPI, NSE or CA19-9 are above the reference level and indicates the subject is suffering from lung cancer.

14. The method of claim 1, wherein the lung cancer treatment regimen comprises administering at least one of surgery, radiotherapeutic therapy, radiotherapeutic treatments, chemotherapy, targeted therapy or combinations thereof to the subject.

15. The method of claim 1, wherein the lung monitoring regimen comprises at least one of conducting a chest computed tomography (CT) scan, a positron emission tomography (PET) scan, measuring lung function and determining MK, TFPI, NSE and CA19-9 levels at periodic intervals.

16. The method of claim 1, wherein determining the level of MK, TFPI, NSE and CA19-9 involves the step of contacting the biological sample with at least one antibody selected from the group consisting of: an antibody that specifically binds to MK, an antibody that specifically binds to TFPI, an antibody that specifically binds to NSE, an antibody that specifically binds to CA19-9 and combinations thereof.

17. The method of claim 1, wherein determining the level of MK, TFPI, NSE and CA19-9 involves the step of assaying the biological sample for MK, TFPI, NSE and CA19-9 by an immunoassay that employs at least one capture antibody and at least one antibody labeled with a detectable label, which generates a signal, and comprises comparing a signal generated by the detectable label as a direct or indirect indication of the amount of MK, TFPI, NSE and CA19-9 in the biological sample, wherein the capture antibody and the antibody labeled with a detectable label comprise:
a. at least one capture antibody that specifically binds to MK and at least one antibody labeled with a detectable label;
b. at least one capture antibody that specifically binds to TFPI and at least one antibody labeled with a detectable label;
c. at least one capture antibody that specifically binds to NSE and at least one antibody labeled with a detectable label; and
d. at least one capture antibody that specifically binds to CA19-9 and at least one antibody labeled with a detectable label.

18. The method of claim 11, wherein the immunological method comprises:
(a) measuring the levels of MK by:
(i) contacting the test sample with at least one capture antibody, wherein the capture antibody binds to an epitope on MK or a fragment of MK to form a capture antibody-MK antigen complex;
(ii) contacting the capture antibody-MK antigen complex with at least one detection antibody comprising a detectable label, wherein the detection antibody binds to an epitope on MK that is not bound by the capture antibody and forms a capture antibody-MK antigen-detection antibody complex; and
(iii) determining the MK levels in the test sample based on the signal generated by the detectable label in the capture antibody-MK-9 antigen-detection antibody complex formed in (a)(ii);
(b) measuring the levels of TFPI by:
(i) contacting the test sample with at least one capture antibody, wherein the capture antibody binds to an epitope on TFPI or a fragment of TFPI to form a capture antibody-TFPI antigen complex;
(ii) contacting the capture antibody-TFPI antigen complex with at least one detection antibody comprising a detectable label, wherein the detection antibody binds to an epitope on TFPI that is not bound by the capture antibody and forms a capture antibody-TFPI antigen-detection antibody complex; and
(iii) determining the TFPI levels in the test sample based on the signal generated by the detectable label in the capture antibody-TFPI antigen-detection antibody complex formed in (b)(ii);
(c) measuring the levels of NSE by:
(i) contacting the test sample with at least one capture antibody, wherein the capture antibody binds to an epitope on NSE or a fragment of NSE to form a capture antibody-NSE antigen complex;
(ii) contacting the capture antibody-NSE antigen complex with at least one detection antibody comprising a detectable label, wherein the detection antibody binds to an epitope on NSE that is not bound by the capture antibody and forms a capture antibody-NSE antigen-detection antibody complex; and
(iii) determining the NSE levels in the test sample based on the signal generated by the detectable label in the capture antibody-NSE antigen-detection antibody complex formed in (c)(ii); and
(d) measuring the levels of CA19-9 by:
(i) contacting the test sample with at least one capture antibody, wherein the capture antibody binds to an epitope on CA19-9 or a fragment of CA19-9 to form a capture antibody-CA19-9 antigen complex;
(ii) contacting the capture antibody-CA19-9 antigen complex with at least one detection antibody comprising a detectable label, wherein the detection antibody binds to an epitope on CA19-9 that is not bound by the capture antibody and forms a capture antibody-CA19-9 antigen-detection antibody complex; and
(iii) determining the CA19-9 levels in the test sample based on the signal generated by the detectable label in the capture antibody-CA19-9 antigen-detection antibody complex formed in (d)(ii).

19. The method of claim 16, 17 or 18, wherein the antibody is selected from the group consisting of: a polyclonal antibody, a monoclonal antibody, a human antibody, an immunoglobulin molecule, a disulfide linked Fv, a monoclonal antibody, an affinity matured, a scFv, a chimeric antibody, a single domain antibody, a CDR-grafted antibody, a diabody, a humanized antibody, a multispecific antibody, a Fab, a dual specific antibody, a DVD, a Fab′, a bispecific antibody, a F(ab′)2, and a Fv.

20. A method of providing a diagnosis of a subject having lung cancer, the method comprising the steps of:
a. obtaining a biological sample from a subject;
b. determining the levels of midkine (MK), tissue factor pathway inhibitor (TFPI), neuron-specific enolase (NSE) and carbohydrate antigen 19-9 (CA19-9) in the biological sample from the subject;
c. determining the number of pack years of smoking of the subject;
d. comparing the levels of MK, TFPI, NSE and CA19-9 in the biological sample to reference levels of MK, TFPI, NSE and CA19-9;
e. comparing the number of pack years of smoking by the subject to a reference level of pack years; and
f. providing a diagnosis of a subject having lung cancer if the levels of MK, TFPI, NSE and CA19-9 in the biological sample are greater than the reference levels of MK, TFPI, NSE and CA19-9 and the number of pack years of smoking by the subject is greater than the reference level of pack years.

21. The method of claim 20, wherein the lung cancer is early lung cancer.

22. The method of claim 21, wherein the early lung cancer is Stage I or Stage II non-small cell lung cancer or limited stage small cell lung cancer.

23. The method of claim 20, wherein determining the level of MK, TFPI, NSE and CA19-9 involves the step of contacting the biological sample with at least one antibody selected from the group consisting of: an antibody that specifically binds to MK, an antibody that specifically binds to TFPI, an antibody that specifically binds to NSE, an antibody that specifically binds to CA19-9 and combinations thereof.

24. The method of claim 20, wherein determining the level of MK, TFPI, NSE and CA19-9 involves the step of assaying the biological sample for MK, TFPI, NSE and CA19-9 by an immunoassay that employs at least one capture antibody and at least one antibody labeled with a detectable label, which generates a signal, and comprises comparing a signal generated by the detectable label as a direct or indirect indication of the amount of MK, TFPI, NSE and CA19-9 in the biological sample, wherein the capture antibody and the antibody labeled with a detectable label comprise:
a. at least one capture antibody that specifically binds to MK and at least one antibody labeled with a detectable label;
b. at least one capture antibody that specifically binds to TFPI and at least one antibody labeled with a detectable label;
c. at least one capture antibody that specifically binds to NSE and at least one antibody labeled with a detectable label; and
d. at least one capture antibody that specifically binds to CA19-9 and at least one antibody labeled with a detectable label.

25. The method of claim 20, wherein determining the level of MK, TFPI, NSE and CA19-9 involves the step of assaying the biological sample for MK, TFPI, NSE and CA19-9 by an immunoassay wherein the immunoassay comprises:
(a) measuring the levels of MK by:
(i) contacting the test sample with at least one capture antibody, wherein the capture antibody binds to an epitope on MK or a fragment of MK to form a capture antibody-MK antigen complex;
(ii) contacting the capture antibody-MK antigen complex with at least one detection antibody comprising a detectable label, wherein the detection antibody binds to an epitope on MK that is not bound by the capture antibody and forms a capture antibody-MK antigen-detection antibody complex; and
(iii) determining the MK levels in the test sample based on the signal generated by the detectable label in the capture antibody-MK-9 antigen-detection antibody complex formed in (a)(ii);
(b) measuring the levels of TFPI by:
(i) contacting the test sample with at least one capture antibody, wherein the capture antibody binds to an epitope on TFPI or a fragment of TFPI to form a capture antibody-TFPI antigen complex;
(ii) contacting the capture antibody-TFPI antigen complex with at least one detection antibody comprising a detectable label, wherein the detection antibody binds to an epitope on TFPI that is not bound by the capture antibody and forms a capture antibody-TFPI antigen-detection antibody complex; and
(iii) determining the TFPI levels in the test sample based on the signal generated by the detectable label in the capture antibody-TFPI antigen-detection antibody complex formed in (b)(ii);
(c) measuring the levels of NSE by:
(i) contacting the test sample with at least one capture antibody, wherein the capture antibody binds to an epitope on NSE or a fragment of NSE to form a capture antibody-NSE antigen complex;
(ii) contacting the capture antibody-NSE antigen complex with at least one detection antibody comprising a detectable label, wherein the detection antibody binds to an epitope on NSE that is not bound by the capture antibody and forms a capture antibody-NSE antigen-detection antibody complex; and
(iii) determining the NSE levels in the test sample based on the signal generated by the detectable label in the capture antibody-NSE antigen-detection antibody complex formed in (c)(ii); and
(d) measuring the levels of CA19-9 by:
(i) contacting the test sample with at least one capture antibody, wherein the capture antibody binds to an epitope on CA19-9 or a fragment of CA19-9 to form a capture antibody-CA19-9 antigen complex;
(ii) contacting the capture antibody-CA19-9 antigen complex with at least one detection antibody comprising a detectable label, wherein the detection antibody binds to an epitope on CA19-9 that is not bound by the capture antibody and forms a capture antibody-CA19-9 antigen-detection antibody complex; and
(iii) determining the CA19-9 levels in the test sample based on the signal generated by the detectable label in the capture antibody-CA19-9 antigen-detection antibody complex formed in (d)(ii).

26. The method of claim 23, 24 or 25, wherein the antibody is selected from the group consisting of: a polyclonal antibody, a monoclonal antibody, a human antibody, an immunoglobulin molecule, a disulfide linked Fv, a monoclonal antibody, an affinity matured, a scFv, a chimeric antibody, a single domain antibody, a CDR-grafted antibody, a diabody, a humanized antibody, a multispecific antibody, a Fab, a dual specific antibody, a DVD, a Fab′, a bispecific antibody, a F(ab′)2, and a Fv.

27. The method of claim 20, wherein the diagnosis is confirmed by at least one of a lung biopsy, a magnetic resonance image (MRI), a CT scan, a positron emission tomography (PET) scan or combinations thereof.

28. A method for the diagnosis, prognosis and/or risk stratification of lung cancer in a subject having or suspected of lung cancer, the method comprising the step of detecting increased levels of midkine (MK), tissue factor pathway inhibitor (TFPI), neuron-specific enolase (NSE) and carbohydrate antigen 19-9 (CA19-9) and an increased amount of pack years of smoking by the subject relative to a control subject not having lung cancer.

29. The method of claim 20 or 28, further comprising administering a lung cancer treatment regimen.

30. A method of determining whether a subject is suffering from early lung cancer or benign lung disease, the method comprising the steps of:
a. obtaining a biological sample from a subject;
b. determining the levels of midkine (MK), tissue factor pathway inhibitor (TFPI), neuron-specific enolase (NSE) and carbohydrate antigen 19-9 (CA19-9) in the biological sample from the subject;
c. determining the number of pack years of smoking of the subject;
d. comparing the levels of MK, TFPI, NSE and CA19-9 in the biological sample to reference levels of MK, TFPI, NSE and CA19-9;
e. comparing the number of pack years of smoking by the subject to a reference level of pack years; and
f. providing a diagnosis of a subject having early lung cancer if the levels of in the MK, TFPI, NSE and CA19-9 in the biological sample are greater than the reference levels of MK, TFPI, NSE and CA19-9 and the number of pack years of the subject are greater than the reference level of pack years or a diagnosis of a subject having benign disease if the levels of MK, TFPI, NSE and CA19-9 in the biological sample are equal or less than the reference levels of MK, TFPI, NSE and CA19-9 and the number of pack years of the subject are equal or less than the reference level of pack years.

31. The method of claim 30, wherein the early lung cancer is Stage I or Stage II non-small cell lung cancer or limited stage small cell lung cancer.

32. The method of claim 30, wherein the reference levels of MK, TFPI, NSE and CA19-9 are the MK, TFPI, NSE and CA19-9 cutoff values determined by a receiver operating curve (ROC) analysis from biological samples of a patient group.

33. The method of claim 30, wherein the reference levels of MK, TFPI, NSE and CA19-9 are the MK, TFPI, NSE and CA19-9 cutoff values determined by a quartile analysis of biological samples of a patient group.

34. The method of claim 30, wherein the MK, TFPI, NSE and CA19-9 reference level is higher than or equal to 0.05 ng/mL, 0.06 ng/mL, 0.07 ng/mL, 0.08 ng/mL, 0.09 ng/mL, 0.10 ng/mL, 0.20 ng/mL, 0.30 ng/mL or 0.40 ng/mL in serum for MK in combination with levels higher than or equal 20 pg/mL, 21 pg/mL, 22 pg/mL, 23 pg/mL, 24 pg/mL, 25 pg/mL, 26 pg/mL, 27 pg/mL, 28 pg/mL, 29 pg/mL, 30 pg/mL, 31 pg/mL, 32 pg/mL, 33 pg/mL, 34 pg/mL, 35 pg/mL, 36 pg/mL, 37 pg/mL, 38 pg/mL, 39 pg/mL or 40 pg/mL in serum for TFPI, levels higher than or equal to 1 ng/mL, 2 ng/mL, 3 ng/mL, 4 ng/mL or 5 ng/mL in serum for NSE and levels higher than or equal to 10 U/mL, 11 U/mL, 12 U/mL, 13 U/mL, 14 U/mL, 15 U/mL, 16 U/mL, 17 U/mL, 18 U/mL, 19 U/mL, 30 U/mL, 31 U/mL, 32 U/mL, 33 U/mL, 34 U/mL, 35 U/mL, 36 U/mL or 37 U/mL in serum for CA19-9.

35. The method of claim 30, further comprising determining the level of at least one additional biomarker of lung cancer in the biological sample selected from the group consisting of: nectin-4, cytokeratin 19-fragment 21-1 (CYFRA 21-1), carcinoembryonic antigen (CEA), progastrin releasing peptide (proGRP), carbohydrate antigen 125 (CA125), tissue polypeptide specific antigen (TPS), cancer antigen 15-3 (CA15-3), squamous cell carcinoma antigen (CCA), Helicobacter pylori, parainfluenza and combinations thereof, and comparing the level of the at least one additional biomarker of lung cancer to a reference level for the at least one biomarker of lung cancer.

36. The method of claim 30, wherein the subject is a human.

37. The method of claim 30, wherein the biological sample of a subject is selected from a tissue sample, bodily fluid, whole blood, plasma, serum, urine, bronchoalveolar lavage fluid, and a cell culture suspension or fraction thereof.

38. The method of claim 30, wherein the biological sample of a subject is blood plasma or blood serum.

39. The method of claim 30, wherein determining the levels of MK, TFPI, NSE and CA19-9 comprises an immunological method with molecules specifically binding to MK, TFPI, NSE or CA19-9.

40. The method of claim 39, wherein the molecules specifically binding to MK, TFPI, NSE and CA19-9 comprises at least one antibody capable of specifically binding MK, TFPI, NSE or CA19-9.

41. The method of claim 34, wherein levels MK, TFPI, NSE or CA19-9 are above the reference level and indicates the subject is suffering from lung cancer.

42. The method of claim 30, further comprising the step of administering a lung cancer treatment regimen to the subject identified as having lung cancer or a lung monitoring regimen to the subject identified as at risk of having lung cancer.

43. The method of claim 42, wherein the lung cancer treatment regimen comprises administering at least one of surgery, radiotherapeutic therapy, radiotherapeutic treatments, chemotherapy, targeted therapy or combinations thereof to the subject.

44. The method of claim 30, wherein determining the level of MK, TFPI, NSE and CA19-9 involves the step of contacting the biological sample with at least one antibody selected from the group consisting of: an antibody that binds to MK, an antibody that binds to TFPI, an antibody that binds to NSE, an antibody that binds to CA19-9 and combinations thereof.

45. The method of claim 30, wherein determining the level of MK, TFPI, NSE and CA19-9 involves the step of assaying the biological sample for MK, TFPI, NSE and CA19-9 by an immunoassay that employs at least one capture antibody and at least one antibody labeled with a detectable label, which generates a signal, and comprises comparing a signal generated by the detectable label as a direct or indirect indication of the amount of MK, TFPI, NSE and CA19-9 in the biological sample, wherein the capture antibody and the antibody labeled with a detectable label comprise:
a. at least one capture antibody that specifically binds to MK and at least one antibody labeled with a detectable label;
b. at least one capture antibody that specifically binds to TFPI and at least one antibody labeled with a detectable label;
c. at least one capture antibody that specifically binds to NSE and at least one antibody labeled with a detectable label; and
d. at least one capture antibody that specifically binds to CA19-9 and at least one antibody labeled with a detectable label.

46. The method of claim 39, wherein the immunological method comprises:
(a) measuring the levels of MK by:
(i) contacting the test sample with at least one capture antibody, wherein the capture antibody binds to an epitope on MK or a fragment of MK to form a capture antibody-MK antigen complex;
(ii) contacting the capture antibody-MK antigen complex with at least one detection antibody comprising a detectable label, wherein the detection antibody binds to an epitope on MK that is not bound by the capture antibody and forms a capture antibody-MK antigen-detection antibody complex; and
(iii) determining the MK levels in the test sample based on the signal generated by the detectable label in the capture antibody-MK-9 antigen-detection antibody complex formed in (a)(ii);
(b) measuring the levels of TFPI by:
(i) contacting the test sample with at least one capture antibody, wherein the capture antibody binds to an epitope on TFPI or a fragment of TFPI to form a capture antibody-TFPI antigen complex;
(ii) contacting the capture antibody-TFPI antigen complex with at least one detection antibody comprising a detectable label, wherein the detection antibody binds to an epitope on TFPI that is not bound by the capture antibody and forms a capture antibody-TFPI antigen-detection antibody complex; and
(iii) determining the TFPI levels in the test sample based on the signal generated by the detectable label in the capture antibody-TFPI antigen-detection antibody complex formed in (b)(ii);
(c) measuring the levels of NSE by:
(i) contacting the test sample with at least one capture antibody, wherein the capture antibody binds to an epitope on NSE or a fragment of NSE to form a capture antibody-NSE antigen complex;
(ii) contacting the capture antibody-NSE antigen complex with at least one detection antibody comprising a detectable label, wherein the detection antibody binds to an epitope on NSE that is not bound by the capture antibody and forms a capture antibody-NSE antigen-detection antibody complex; and
(iii) determining the NSE levels in the test sample based on the signal generated by the detectable label in the capture antibody-NSE antigen-detection antibody complex formed in (c)(ii); and
(d) measuring the levels of CA19-9 by:
(i) contacting the test sample with at least one capture antibody, wherein the capture antibody binds to an epitope on CA19-9 or a fragment of CA19-9 to form a capture antibody-CA19-9 antigen complex;
(ii) contacting the capture antibody-CA19-9 antigen complex with at least one detection antibody comprising a detectable label, wherein the detection antibody binds to an epitope on CA19-9 that is not bound by the capture antibody and forms a capture antibody-CA19-9 antigen-detection antibody complex; and
(iii) determining the CA19-9 levels in the test sample based on the signal generated by the detectable label in the capture antibody-CA19-9 antigen-detection antibody complex formed in (d)(ii).

47. The method of claim 44, 45 or 46, wherein the antibody is selected from the group consisting of: a polyclonal antibody, a monoclonal antibody, a human antibody, an immunoglobulin molecule, a disulfide linked Fv, a monoclonal antibody, an affinity matured, a scFv, a chimeric antibody, a single domain antibody, a CDR-grafted antibody, a diabody, a humanized antibody, a multispecific antibody, a Fab, a dual specific antibody, a DVD, a Fab′, a bispecific antibody, a F(ab′)2, and a Fv.

48. The method of claim 30, wherein the diagnosis is confirmed by at least one of a lung biopsy, a magnetic resonance image (MRI), a CT scan, a positron emission tomography (PET) scan or combinations thereof.

49. A kit for performing the method of claim 1, the kit comprising:
a. a reagent capable of specifically binding to MK, a reagent capable of specifically binding to TFPI, a reagent capable of specifically binding to NSE and a reagent capable of specifically binding to CA19-9 to quantify the levels of MK, TFPI, NSE and CA19-9 in the biological sample of a subject;
b. a reference standard indicating reference levels of MK, TFPI, NSE and CA19-9; and
c. a reference standard indicating a reference level of pack years.

50. The kit of claim 49, further comprising at least one additional reagent capable of specifically binding at least one additional biomarker of nectin-4, cytokeratin 19-fragment 21-1 (CYFRA 21-1), carcinoembryonic antigen (CEA), progastrin releasing peptide (proGRP), carbohydrate antigen 125 (CA125), tissue polypeptide specific antigen (TPS), cancer antigen 15-3 (CA15-3), squamous cell carcinoma antigen (CCA), Helicobacter pylori and/or parainfluenza in the biological sample to quantify the concentration of the at least one additional biomarker in the biological sample, and a reference standard indicating a reference level of the at least one additional biomarker nectin-4, cytokeratin 19-fragment 21-1 (CYFRA 21-1), carcinoembryonic antigen (CEA), progastrin releasing peptide (proGRP), carbohydrate antigen 125 (CA125), tissue polypeptide specific antigen (TPS), cancer antigen 15-3 (CA15-3), squamous cell carcinoma antigen (CCA), Helicobacter pylori and/or parainfluenza of in the biological sample.
